(19) United States
(12) Patent Application Publication
(10) Pub. No.: US 2013/0146053 A1
(43) Pub. Date: Jun. 13, 2013

(54) VENTILATION CIRCUIT ADAPTOR AND PROXIMAL AEROSOL DELIVERY SYSTEM

(71) Applicants: Jan Mazela, Poznan (PL); Christopher R. Henderson, Solana Beach, CA (US); Hope M. Miller, Philadelphia, PA (US)

(72) Inventors: Jan Mazela, Poznan (PL); Christopher R. Henderson, Solana Beach, CA (US); Hope M. Miller, Philadelphia, PA (US)

(73) Assignee: Discovery Laboratories, Inc., Warrington, PA (US)

(21) Appl. No.: 13/668,541

(22) Filed: Nov. 5, 2012

Related U.S. Application Data
(63) Continuation-in-part of application No. 12/922,981, filed on Sep. 16, 2010.
(60) Provisional application No. 61/555,774, filed on Nov. 4, 2011.
(60) Provisional application No. 61/069,850, filed on Mar. 17, 2008.
(60) Provisional application No. 61/076,442, filed on Jun. 27, 2008.

(51) Int. Cl. A61M 16/10; B23P 11/00

(52) U.S. Cl. 128/203.12; 137/15.01

(57) ABSTRACT

An adaptor for delivering an active agent to a patient with concomitant positive pressure ventilation includes an aerosol flow channel having an aerosol inlet port and a patient interface port, and defining an aerosol flow path from the aerosol inlet port to and through the patient interface port; and a ventilation gas flow channel in fluid communication with the aerosol flow channel and having a gas inlet port and a gas outlet port, and defining a ventilation gas flow path from the gas inlet port to and through the gas outlet port, wherein the ventilation gas flow path is at least partially offset from the aerosol flow path and at least partially encircles the aerosol flow path. Systems and methods for delivering an active agent to a patient with concomitant positive pressure ventilation incorporate the adaptor.

### CROSS-REFERENCE TO RELATED APPLICATIONS

This application claims the benefit under 35 U.S.C. §120 of U.S. Provisional Patent Application Ser. No. 61/555,774, filed Nov. 4, 2011, titled Ventilation Circuit Adaptor And Proximal Aerosol Delivery System, which is a Continuation-In-Part (OP) of U.S. patent application Ser. No. 12/922,981, filed Sep. 16, 2010 which is a U.S. national phase application of International (PCT) Patent Application No. PCT/US2009/037409, filed Mar. 17, 2009 and which claims priority benefit of U.S. Provisional Patent Application Nos. 61/069,850, filed Mar. 17, 2008, titled Ventilation Circuit Adaptor and 61/076,442, filed Jun. 27, 2008, titled Ventilation Circuit Adaptor And Proximal Aerosol Delivery System, the entire disclosures of which are hereby expressly incorporated by reference herein.

### BACKGROUND

1. Field of Invention

This invention relates to pulmonary therapy and ventilatory support of pulmonary function. In particular, the invention is directed to an aerosol delivery system and a ventilation circuit adaptor for pulmonary delivery of aerosolized substances and/or for other therapeutic and/or diagnostic purposes, in combination with noninvasive or invasive respiratory ventilation support.

2. Description of Related Art

Various patents, patent publications and scientific articles may be referred to throughout the specification. The contents of each of these documents are incorporated by reference herein, in their entireties.

Patients, both adult and infants, in respiratory failure or those with respiratory dysfunction are typically mechanically ventilated in order to provide suitable rescue and prophylactic therapy. Respiratory failure in adults or infants can be caused by any condition relating to poor breathing, muscle weakness, abnormality of lung tissue, abnormality of the chest wall, and the like. Additionally, pre- and full-term infants born with a respiratory dysfunction, such as respiratory distress syndrome (RDS), meconium aspiration syndrome (MAS), persistent pulmonary hypertension (PPHN), acute respiratory distress syndrome (ARDS), pheumocystis carinii pneumonia (PCP), transient tachypnea of the newborn (TTN) and the like often require prophylactic or rescue respiratory support. In addition to respiratory support, infants suffering from, or at risk of RDS are often treated with exogenous surfactant, which improves gas exchange and has had a dramatic impact on mortality. Typically, the exogenous material is delivered as a liquid bolus to the central airways via a catheter introduced through an endotracheal tube. Infants born at 28 weeks or less are almost universally intubated and mechanically ventilated. There is a significant risk of failure during the process of intubation and a finite chance of causing damage to the upper trachea, laryngeal folds and surrounding tissue. Mechanical ventilation over a prolonged time, particularly where elevated oxygen tensions are employed, can also lead to acute lung damage. If ventilation and oxygen is required for prolonged periods of time and/or if the ventilator is not sufficiently managed, the clinical consequences can include bronchopulmonary dysplasia, chronic lung disease, pulmonary hemorrhage, intraventricular hemorrhage, and periventricular leukomalacia.

Infants born of larger weight or gestational age who are not overtly at risk of developing respiratory distress syndrome, or infants who have completed treatment for respiratory distress syndrome can be supported by noninvasive means. Attempts were made to administer liquid surfactant without intubation: to the posterior pharynx through the catheter, with spontaneously breathing infant [1], or to the pharynx through the laryngeal mask with transient positive pressure ventilation (PPV) [2]. Another non-invasive approach is nasal continuous positive airway pressure ventilation (nCPAP or CPAP). CPAP is a means to provide voluntary ventilator support while avoiding the invasive procedure of intubation. Nasal CPAP is widely accepted among clinicians as a less invasive mode of ventilatory support for preterm newborns with mild/moderate RDS. CPAP has been demonstrated to be effective in increasing functional residual capacity (FRC) by stabilizing and improving alveolar function [3], and in dilating the larynx [4]. Based on animal work, CPAP in combination with surfactant therapy has been also shown to minimize the risk for bronchopulmonary dysplasia (BPD) development among preterm baboons [5]. Randomized clinical trials focused on the use of nCPAP in the prophylaxis of RDS did show the benefit of nCPAP after instillation of surfactant via endotracheal tube [6, 7]. CPAP provides humidified and slightly over-pressurized gas (approximately 5 cm H₂O above atmospheric pressure) to an infant's nasal passageway utilizing nasal prongs or a tight fitting nasal mask. CPAP also has the potential to provide successful treatment for adults with various disorders including chronic obstructive pulmonary disease (COPD), sleep apnea, acute lung injury (ALI)/ARDS and the like.

A typical ventilatory circuit for administering positive pressure ventilation includes a positive pressure generator connected by tubing to a patient interface, such as a mask, nasal prongs, or an endotracheal tube, and an exhalation path, such as tubing that allows discharge of the expired gases, e.g., to the ventilator or to an underwater receptacle as for “bubble” CPAP. The inspiratory and expiratory tubes are typically connected to the patient interface via a “Y” connector, which contains a port for attaching each of the inspiratory and expiratory tubes, as well as a port for the patient interface and, typically, a port for attaching a pressure sensor. In a closed system, such as with use of a tight-fitting mask or endotracheal tube, administration of other pulmonary treatment, e.g., pulmonary surfactant, or diagnosis generally requires temporary disconnection of the ventilatory support while the pulmonary treatment is administered or the diagnosis is conducted.

Recent efforts have focused on delivery of surfactant and/or other active agents in an aerosolized form, in order to enhance delivery and/or avoid or minimize the trauma of prolonged invasive mechanical ventilation. However, if the patient is receiving ongoing ventilatory support, administration of aerosolized active agents may necessitate interruption of the ventilatory support while the aerosol is administered. As a result, attempts have been made to deliver aerosolized active agents simultaneously with noninvasive positive pressure. For instance, Berggren et al. (Acta Poediatr. 2000, 89:460-464) attempted to delivery pulmonary surfactant simultaneously with CPAP, but were unsuccessful due to the lack of sufficient quantities of surfactant reaching the lungs.

U.S. Patent publication 2006/0120968 by Niven et al. describes the concomitant delivery of positive pressure ventilation and active aerosolized agents, including pulmonary surfactants. Delivery was reported to be accomplished through the use of a device and system that was designed to improve the flow and direction of aerosols to the patient interface while substantially avoiding dilution by the ventilation gas stream. The system employed an aerosol conditioning chamber and a uniquely-shaped connector for directing the aerosol and the ventilation gas.

U.S. Pat. No. 7,201,167 to Fink et al., describes a method of treating a disease involving surfactant deficiency or dysfunction by providing aerosolized lung surfactant composition into the gas flow within a CPAP system. As shown in FIGS. 1 and 6 of the Fink et al. patent, the aerosol is carried by air coming from a flow generator wherein the aerosol is being diluted with the air.

Typically, a constant flow CPAP/ventilator circuit used for breathing support consists of an inspiratory arm, a patient interface, an expiratory arm and a source of positive end expiratory pressure (PEEP valve or column of water). Currently, aerosol generator manufacturers place nebulizers within the inspiratory arm of the CPAP/ventilator tubing circuit. This can potentially lead to aerosol dilution and decrease in aerosol concentration (see U.S. Pat. No. 7,201,167 to Fink et al.). Aerosol dilution is caused by much higher flows in the CPAP/ventilator circuit as compared to the peak inspiratory flow (PIF) of treated patients. Placement of the nebulizer between ‘Y’ connector and endotracheal tube (ET) or other patient interface as proposed by Fink et al. [11] account for significant increase in dead space depraving patient from appropriate ventilation.

To overcome the deficiencies of the prior art, the inventors developed a special adaptor which enables sufficient separation of the aerosol or gasified agent flow from the ventilation flow maintaining optimized ventilation as well as a novel aerosol delivery system.

All references cited herein are incorporated herein by reference in their entireties.

### BRIEF SUMMARY

One aspect of the invention features a respiratory ventilation adaptor useful for delivery of a fluid, e.g., an aerosolized or a gasified active agent, to a patient with concomitant positive pressure ventilation. The adaptor comprises: (a) an aerosol flow channel comprising an aerosol inlet port and a patient interface port, and defining an aerosol flow path from the aerosol inlet port to and through the patient interface port; and (b) a ventilation gas flow channel in fluid communication with the aerosol flow channel, comprising a gas inlet port and a gas outlet port, and defining a ventilation gas flow path from the gas inlet port to and through the gas outlet port; wherein the ventilation gas flow path is at least partially offset from the aerosol flow path and at least partially encircles the aerosol flow path.

The adaptor can further comprise a sensor port, for example, a pressure sensor port. The adaptor may also further comprise a valve at the aerosol inlet port. In one embodiment, the valve is a slit or cross-slit valve. In various embodiments, the valve is sufficiently flexible to allow introduction of instruments, catheters, tubes, or fibers into and through the aerosol flow channel and the patient interface port, while maintaining positive ventilatory pressure. The adaptor may also further comprise a removable cap covering the aerosol inlet port. The cap may also be tethered. The adaptor may further comprise a one-way valve at the aerosol outlet port.

In certain embodiments, the aerosol flow channel defines a substantially straight aerosol flow path, whereas in other embodiments, the aerosol flow channel defines a curved or angled aerosol flow path. The aerosol flow channel is of substantially the same cross-sectional area throughout its length, or it can be of greater cross sectional area at the aerosol inlet port than it is at the patient interface port. In certain embodiments, the fluid communication between the aerosol flow channel and the ventilation gas flow channel can be provided by an aperture.

In certain embodiments, the ventilation gas flow channel is adapted to form a chamber that includes the gas inlet port, the gas outlet port and the patient interface port, wherein the aerosol flow channel is contained within the chamber and extends from the aerosol inlet port at one end of the chamber, through the chamber to an aerosol outlet port within the chamber and recessed from the patient interface port at the opposite end of the chamber, wherein the aerosol flow channel is of sufficient length to extend beyond the gas inlet and outlet ports. In particular embodiments the aerosol outlet port is recessed from the patient interface port by about 8 millimeters or more. In other particular embodiments, the volume within the chamber between the aerosol outlet port and the patient interface port is about 1.4 milliliters or more.

Another aspect of the invention features a system for delivery of a fluid, e.g., an aerosolized or gasified active agent, to a patient with concomitant positive pressure ventilation, the system comprising: (a) a positive pressure ventilation circuit comprising a positive pressure generator for producing pressurized ventilation gas and a delivery means for delivering the pressurized ventilation gas to the patient and for directing exhalation gases from the patient; (b) an aerosol generator for producing the aerosolized active agent; and (c) a patient interface for delivering the ventilation gas and the aerosolized active agent to the patient; wherein the positive pressure ventilation circuit and the aerosol generator are connected to the patient interface through a respiratory ventilation adaptor comprising: (i) an aerosol flow channel having an aerosol inlet port and a patient interface port, and defining an aerosol flow path from the aerosol inlet port to and through the patient interface port; and (ii) a ventilation gas flow channel in fluid communication with the aerosol flow channel, comprising a gas inlet port and a gas outlet port, and defining a ventilation gas flow path from the gas inlet port to an through the gas outlet port; wherein the ventilation gas flow path is at least partially offset from the aerosol flow path and at least partially encircles the aerosol flow path.

The adaptor may further comprise a sensor port connected to a sensor, such as, for example, a pressure sensor, as well as a valve at the aerosol inlet port. In embodiments of the system, connection of the aerosol generator to the adaptor causes the valve to open, and disconnection of the aerosol generator from the adaptor causes the valve to close. In certain embodiments, the valve, when closed, is sufficiently flexible to allow introduction of instruments, catheters, tubes, or fibers into and through the aerosol flow channel and the patient interface port, while maintaining positive ventilatory pressure. The system may further comprise an adaptor with a removable cap for the aerosol inlet port, for use when the aerosol generator is disconnected from the adaptor. In certain embodiments, the patient interface is not invasive, e.g., is a mask or nasal prongs. In other embodiments, the patient interface is invasive, e.g., an endotracheal tube.

Another aspect of the invention relates to a system for delivery of a propelled fluid, e.g., an aerosolized or gasified active agent, with concomitant positive pressure ventilation to a patient, the system comprising: a) a respiratory ventilation adaptor adapted to communicate with a positive pressure ventilation circuit, an aerosol generator or a source of active agent capable of producing an aerosolized or gasified active agent and a patient interface; and b) an auxiliary circuit adapted to communicate with a delivery conduit for delivering a pressurized ventilation gas to the respiratory ventilation adaptor, wherein the auxiliary circuit comprises a first auxiliary conduit adapted to connect the delivery conduit and an aerosol entrainment chamber and a second auxiliary conduit adapted to connect the aerosol entrainment chamber and the respiratory ventilation adaptor, wherein the first auxiliary conduit is adapted to accommodate a portion of the pressurized ventilation gas to be removed from a main flow of the pressurized ventilation gas directed toward the respiratory ventilation adaptor, and to enable delivery of the portion of the pressurized ventilation gas to the aerosol entrainment chamber for combining with the aerosolized or gasified active agent to form the propelled fluid and the second auxiliary conduit is adapted to enable delivery of the propelled fluid to the respiratory ventilation adaptor.

Yet another aspect of the invention relates to a method of delivery of a propelled aerosolized active agent with concomitant positive pressure ventilation to a patient, the method comprising: a) providing a positive pressure ventilation circuit comprising a positive pressure generator for producing pressurized ventilation gas and a delivery conduit for delivering the pressurized ventilation gas to the patient and for directing exhalation gases from the patient; b) providing an aerosol generator for producing an aerosolized active agent; c) providing a patient interface for delivering the ventilation gas and the aerosolized active agent to the patient; d) providing a respiratory ventilation adaptor in communication with the positive pressure ventilation circuit, the aerosol generator and the patient interface; e) providing an aerosol entrainment chamber in communication with the aerosol generator; f) providing an auxiliary circuit in connection with the delivery conduit for delivering the pressurized ventilation gas to the patient, wherein the auxiliary circuit comprises a first auxiliary conduit connecting the delivery conduit and the aerosol entrainment chamber and a second auxiliary conduit connecting the aerosol entrainment chamber and the respiratory ventilation adaptor; g) removing a portion of the pressurized ventilation gas from a main flow of the pressurized ventilation gas directed toward the respiratory ventilation adaptor to the first auxiliary conduit and directing the portion of the pressurized ventilation gas to the aerosol entrainment chamber and thereby combining the portion with the aerosolized active agent to form a propelled aerosolized active agent; h) directing the propelled aerosolized active agent to the second auxiliary conduit and thereby deliver the propelled aerosolized active agent to the respiratory ventilation adaptor; and i) providing the propelled aerosolized active agent and the pressurized ventilation gas to the patient interface and thereby deliver the ventilation gas and the propelled aerosolized active agent to the patient.

Yet another aspect of the invention is an improvement to a method of delivery of an aerosolized active agent with concomitant positive pressure ventilation to a patient in need of pulmonary lung surfactant, the improvement comprising diverting a portion of pressurized ventilation gas directed to the patient to be combined with a concentrated aerosolized active agent in a chamber and using the portion of the pressurized ventilation gas as a carrier (sheath) gas for delivery of the aerosolized active agent to the patient.

Yet another aspect of the invention is a method for delivering an aerosolized active agent to a patient with concomitant positive pressure ventilation, the method comprising: a) providing a positive pressure ventilation circuit comprising a positive pressure generator for producing a pressurized ventilation gas and a delivery conduit for delivering an amount of the pressurized ventilation gas to the patient and for directing a flow of exhalation gas from the patient; b) providing an aerosol generator for producing the aerosolized active agent; c) providing a patient interface for delivering the ventilation gas, the aerosolized active agent or the mixture thereof to the patient; d) connecting the positive pressure ventilation circuit and the aerosol generator to the patient interface through an adaptor, the adaptor comprising: i) an aerosol flow channel having an aerosol inlet port and a patient interface port, and defining an aerosol flow path from the aerosol inlet port to and through the patient interface port; and ii) a ventilation gas flow channel in fluid communication with the aerosol flow channel and having a gas inlet port and a gas outlet port, and defining a ventilation gas flow path from the gas inlet port to and through the gas outlet port, wherein the ventilation gas flow path is at least partially offset from the aerosol flow path and at least partially encircles the aerosol flow path; e) providing the pressurized ventilation gas to the patient, wherein the volume of the pressurized ventilation gas is regulated by at least one of the length of the aerosol flow channel and the pressure created by an increased demand for air which is not matched by the aerosol flow; and f) providing an aerosol flow of the aerosolized active agent to a chamber inside the adaptor such that aerosol flow is introduced below the ventilation gas flow channel wherein the aerosol flow is selected to match the patient's inspiratory flow and thereby providing the aerosolized active agent to the patient. Other features and advantages of the invention will be understood by reference to the drawings, detailed description and examples that follow.

In addition, there are various other aspects of Applicants' ventilation circuit adaptors and methods for assembling the ventilation circuit adaptors, and many variations of each of those aspects.

One such aspect is a first ventilation circuit adaptor which includes an aerosol flow chamber, a ventilation gas flow chamber in fluid communication with the aerosol flow chamber, and a funnel-shaped aerosol flow channel adapted to be inserted into and fixedly positioned in the aerosol flow chamber. The aerosol flow chamber has a first end, a second end opposite the first end, a first longitudinal axis, an inner wall spaced apart from and surrounding the first longitudinal axis, an aerosol chamber inlet port located at the first end and having a first chamber cross-sectional area, and a patient interface port located at the second end and having a second chamber cross-sectional area. The ventilation gas flow chamber has a primary end, an other end spaced apart from the primary end, a second longitudinal axis, a ventilation gas inlet port located at the primary end, and a ventilation gas outlet port located at the other end. The second longitudinal axis is at least partly offset from the first longitudinal axis and at least partially encircles the first longitudinal axis. The funnel-shaped aerosol flow channel has a first channel end, and other channel end opposite the first channel end, a channel longitudinal axis, an outer wall spaced apart from and surrounding the channel longitudinal axis, an aerosol channel inlet port located at the first channel end and having a first channel cross-sectional area, and a channel outlet port located at the second channel end and having a second channel cross-sectional area smaller than the first channel cross-sectional area. The channel longitudinal axis of the funnel-shaped aerosol flow channel is coaxial with the first longitudinal axis of the aerosol flow chamber when the funnel-shaped aerosol flow channel is fixedly positioned in the aerosol flow chamber.

In a first variation of the first ventilation circuit adaptor, the first chamber cross-sectional area is greater than the second chamber cross-sectional area.

In another variation of any of the ventilation circuit adaptors discussed in the previous two paragraphs, the channel outlet port extends beyond the gas inlet port and the gas outlet port, and the second channel end is recessed from the patient interface port. In a variation of those variations, the second channel end is recessed from the patient interface port by a distance (L2) sufficient to reduce or prevent the mixing of the ventilation flow with the flow of active agent and to minimize resistance arising from the patient's exhalations. In certain embodiments, that distance is at least 2 mm. In certain embodiments designed for neonatal use, the second channel end is recessed from the patient interface port by at least about 8 millimeters with the chamber volume in the recess being at least about 1.4 milliliters. In certain embodiments designed for older infants, children or adults, the second channel end can be further recessed from the patient interface port, e.g., by at least about 9, 10, 11, 12, 13, 14, 15 or 16 millimeters, with concomitantly increased chamber volume in the recess, e.g., at least about 1.5, 1.6, 1.7, 1.8, 1.9, 2.0, 2.1, 2.2, 2.3, 2.4, 2.5, 2.6, 2.7, 2.8, 2.9 or 3.0 milliliters. In other embodiments, L2 is in a range of about 4 millimeters to about 8.5 millimeters.

A second ventilation circuit adaptor is similar to the first ventilation circuit adaptor or any of the variations discussed above, but also includes a positive interference seal on a portion of the outer wall of the funnel-shaped aerosol flow channel, the positive interference seal adapted to form a positive interference fit with a portion of the inner wall of the aerosol flow chamber. In a variation of any of those adaptors or variations thereof, the positive interference seal includes a ridge protruding from the portion of the outer wall of the funnel-shaped aerosol flow channel.

A third ventilation circuit adaptor is similar to the first ventilation circuit adaptor or any of the variations discussed above, but also includes at least one assembly alignment fixture adapted to prevent rotational movement of the funnel-shaped aerosol channel when it is fixedly positioned in the aerosol flow chamber. In a variation of any of those adaptors or variations thereof, the at least one assembly alignment fixture includes two or more assembly alignment fixtures circumferentially spaced apart from each other by about 60° to about 180°.

In another variation of the third ventilation circuit adaptor or any of the variations thereof, the at least one assembly alignment fixture includes: at least one aperture or recess in the inner wall of the aerosol flow chamber, and at least one snap-in catch on the outer wall of the funnel-shaped aerosol flow channel adapted to lock with the at least one aperture or recess.

A fourth ventilation circuit adaptor is similar to the first, second, or third ventilation circuit adaptors or any of the variations thereof discussed above, but includes a pressure sensor port in fluid communication with the aerosol flow chamber below the gas inlet port and the gas outlet port.

A fifth ventilation circuit adaptor is similar to the first, second, third, or fourth ventilation circuit adaptors or any of the variations thereof as discussed above, but also includes a removable stopper or cap tethered to an outer surface of the ventilation circuit adaptor and adapted to close the aerosol chamber inlet port.

A sixth ventilation circuit adaptor is similar to the first, second, third, fourth, or fifth ventilation circuit adaptors or any of the variations thereof discussed above, but also includes a reducer having a second inner diameter smaller than a first inner diameter of the patient interface port, wherein the reducer is adjacent to and in fluid communication with the patient interface port and is adapted to receive an aerosol flow from the patient interface port. In a variation of any of those adaptors or variations thereof, a portion of the reducer is connected to the inner wall near the second end of the aerosol flow chamber by a connecting technique selected from a group consisting of ultrasonic welding, gluing, and laser welding.

Another aspect is a ventilation circuit adaptor including an aerosol flow chamber, a ventilation gas flow chamber in fluid communication with the aerosol flow chamber, and a reducer. The aerosol flow chamber has an aerosol inlet port and a patient interface port, and defines an aerosol flow path from the aerosol inlet port to and through the patient interface port having a first inner diameter. The ventilation gas flow chamber has a gas inlet port and a gas outlet port, and defines a ventilation gas flow path from the gas inlet port to and through the gas outlet port, wherein the ventilation gas flow path is at least partially offset from the aerosol flow path and at least partially encircles the aerosol flow path. The ventilation gas flow chamber forms a chamber that includes the inlet port, the gas outlet port and the patient interface port, wherein an aerosol flow channel is contained within the chamber and extends from the aerosol inlet port at one end of the chamber through the chamber to an aerosol outlet port within the chamber and is recessed from the patient interface port at the opposite end of the chamber, wherein the aerosol flow channel has a substantially uniform cross-sectional area and is of a sufficient length to extend beyond the gas inlet and outlet ports. The reducer has a second inner diameter smaller than the first inner diameter of the patient interface port, wherein the reducer is adjacent to and in fluid communication with the patient interface port and is adapted to receive an aerosol flow from the patient interface port.

In a first variation of the apparatus discussed in the previous paragraph, a portion of the reducer is connected to an inner wall of the chamber near the patient interface port by a connecting technique selected from a group consisting of ultrasonic welding, gluing, and laser welding.

Yet another aspect is a method for assembling a ventilation circuit adaptor, which method for assembling includes five steps. The first step is to provide an aerosol flow chamber having a first end, a second end opposite the first end, a first longitudinal axis, an inner wall spaced apart from and surrounding the first longitudinal axis, an aerosol chamber inlet port located at the first end and having a first chamber cross-sectional area, and a patient interface port located at the second end and having a second chamber cross-sectional area. The second step is to provide a ventilation gas flow chamber in fluid communication with the aerosol flow chamber and having a primary end, an other end spaced apart from the primary end, a second longitudinal axis, a ventilation gas inlet port located at the primary end, and a ventilation gas outlet port located at the other end, wherein the second longitudinal axis is at least partially offset from the first longitudinal axis and at least partially encircles the first longitudinal axis. The third step is to provide a funnel-shaped aerosol flow channel adapted to be inserted into and fixedly positioned in the aerosol flow chamber, the funnel-shaped aerosol flow channel having a first channel end, an other channel end opposite the first channel end, a channel longitudinal axis, an outer wall spaced apart from and surrounding the channel longitudinal axis, an aerosol channel inlet port located at the first channel end and having a first channel cross-sectional area, and a channel outlet port located at the second channel end and having a second channel cross-sectional area smaller than the first channel cross-sectional area, wherein the channel longitudinal axis of the funnel-shaped aerosol flow channel is coaxial with the first longitudinal axis of the aerosol flow chamber when the funnel-shaped aerosol flow channel is fixedly positioned in the aerosol flow chamber. The fourth step is to insert the funnel-shaped aerosol flow chamber into the aerosol flow chamber. The fifth step is to fixedly position the funnel-shaped aerosol flow channel in the aerosol flow chamber so that the channel longitudinal axis of the funnel-shaped aerosol flow channel is coaxial with the first longitudinal axis of the aerosol flow chamber.

A second method for assembling a ventilation circuit adaptor is similar to the first method for assembling discussed above, but includes two further steps. The first further step is to provide a positive interference seal on a portion of the outer wall of the funnel-shaped aerosol flow channel, the positive interference seal adapted to form a positive interference fit with a portion of the inner wall of the aerosol flow chamber. The second further step is to form the positive interference fit by the interference seal with a portion of the inner wall of the aerosol flow chamber. In a variation of the second method for assembling, the positive interference seal includes a ridge protruding from the portion of the outer wall of the funnel-shaped aerosol flow channel.

A third method for assembling a ventilation circuit adaptor is similar to the first or second methods for assembling or any variations thereof discussed above, but includes a further step. The further step is to provide at least one assembly alignment fixture adapted to prevent rotational movement of the funnel-shaped aerosol flow channel when it is fixedly positioned in the aerosol flow chamber. In one variation of this method for assembling, the at least one assembly alignment fixture includes: at least one aperture or recess in the inner wall of the aerosol flow chamber, and at least one snap-in catch on the outer wall of the funnel-shaped aerosol flow channel to lock with the at least one aperture or recess.

In a variation of the methods for assembling and the variations thereof discussed in the previous paragraph, the at least one assembly alignment fixture includes two or more assembly alignment fixtures circumferentially spaced apart from each other by about 60° to about 180°.

A fourth method for assembling a ventilation circuit adaptor is similar to the third method for assembling and the variations thereof discussed in the two previous paragraphs, but includes a further step. The further step is to lock the at least one snap-in catch with the at least one aperture or recess.

A fifth method for assembling a ventilation circuit adaptor is similar to the first, second, third, or fourth methods for assembling or any of the variations thereof discussed above, but includes the further step of providing a pressure sensor port in fluid communication with the aerosol flow chamber below the gas inlet port and the gas outlet port.

A sixth method for assembling a ventilation circuit adaptor is similar to the first, second, third, fourth, or fifth methods for assembling or any of the variations thereof discussed above, but includes the further step of providing a removable stopper or cap tethered to an outer surface of the ventilation circuit adaptor and adapted to close the aerosol chamber inlet port.

A seventh method for assembling a ventilation circuit adaptor is similar to the first, second, third, fourth, fifth, or sixth methods for assembling or any of the variations thereof discussed above, but includes a further step. The further step is to provide a reducer having a second inner diameter smaller than a first inner diameter of the patient interface port, wherein the reducer is adjacent to and in fluid communication with the patient interface port and is adapted to receive an aerosol flow from the patient interface port. In a variation of any of those methods for assembling or the variations thereof, a portion of the reducer is connected to the inner wall near the second end of the aerosol flow chamber by a connecting technique selected from a group consisting of ultrasonic welding, gluing, and laser welding.

Yet another aspect is a method for assembling a ventilation circuit adaptor, which method includes four steps. The first step is to provide an aerosol flow chamber having an aerosol inlet port and a patient interface port, and defining an aerosol flow path from the aerosol inlet port to and through the patient interface port having a first inner diameter. The second step is to provide a ventilation gas flow chamber in fluid communication with the aerosol flow chamber and having a gas inlet port and a gas outlet port, and defining a ventilation gas flow path from the gas inlet port to and through the gas outlet port, wherein the ventilation gas flow path is at least partially offset from the aerosol flow path and at least partially encircles the aerosol flow path. The ventilation gas flow chamber forms a chamber that includes the gas inlet port, the gas outlet port and the patient interface port, wherein an aerosol flow channel is contained within the chamber and extends from the aerosol inlet port at one end of the chamber through the chamber to an aerosol outlet port within the chamber and is recessed from the patient interface port at the opposite end of the chamber, wherein the aerosol flow channel has a substantially uniform cross-sectional area and is of a sufficient length to extend beyond the gas inlet and outlet ports. The third step is to provide a reducer having a second inner diameter smaller than the first inner diameter of the patient interface port, wherein the reducer is adjacent to and in fluid communication with the patient interface port and is adapted to receive an aerosol flow from the patient interface port. The fourth step is to connect the reducer to an inner wall of the chamber near the patient interface port.

In a variation of the method for assembling a ventilation circuit adaptor discussed in the above paragraph, the way of connecting the reducer to the inner wall of the chamber near the patient interface port is selected from a group consisting of ultrasonic welding, gluing, and laser welding.

Another method for assembling a ventilation circuit adaptor is similar to the method for assembling and the variations thereof discussed in the above two paragraphs but includes the further step of providing a pressure sensor port in fluid communication with the aerosol flow chamber below the gas inlet port and the gas outlet port.

Yet another method for assembling a ventilation circuit adaptor is similar to the methods for assembling and the variations thereof discussed in the above three paragraphs but includes the further step of providing a removable stopper or cap tethered to an outer surface of the ventilation circuit adaptor and adapted to close the aerosol chamber inlet port.

### BRIEF DESCRIPTION OF SEVERAL VIEWS OF THE DRAWINGS

FIG. 1A is an isometric view of an embodiment of the adaptor of the present invention.

FIGS. 1B and 1C are isometric views of alternative embodiments of the adaptor.

FIG. 2A is a plan view of the front of the adaptor of FIG. 1A.

FIG. 2B is a section view of the adaptor of FIG. 2A, as seen along line 2B-2B.

FIG. 2C is a section view of the adaptor of FIG. 2A as seen along line 2B-2B, showing an alternative internal configuration.

FIG. 2D is a section view of the adaptor of FIG. 2A, as seen along line 2D-2D.

FIG. 3 is an isometric section view of a portion of the adaptor of FIG. 1A.

FIG. 4 is another isometric section view of another portion of the adaptor of FIG. 1A.

FIG. 5A is an isometric view of another embodiment of the adaptor of the present invention.

FIGS. 5B and 5C are isometric views of alternative embodiments of the adaptor.

FIG. 6 is a top view of the adaptor shown in FIG. 5B.

FIG. 7 is a plan view of the front of the adaptor of FIG. 5B.

FIG. 8 illustrates a ventilatory circuit including an adaptor of the type shown in FIG. 1A, 1B, or 1C.

FIG. 9 is a schematic diagram illustrating a proximal aerosol delivery system (PADS).

FIG. 10 a schematic diagram illustrating another embodiment of a proximal aerosol delivery system (PADS) suitable for delivery of multiple substances.

FIG. 11 a schematic diagram illustrating another embodiment of a proximal aerosol delivery system (PADS) suitable for delivery of multiple substances.

FIG. 12A is an isometric view of a component of an additional embodiment of the adaptor shown in FIG. 12C.

FIG. 12B is an isometric view of another component of the additional alternative embodiment of the adaptor shown in FIG. 12C.

FIG. 12C is an isometric view of the additional alternative embodiment of the adaptor comprising the assembly of the components of FIGS. 12A and 12B.

FIG. 13A is a plan view of the front of the adaptor of FIG. 12C with an optional component of a tethered removable stopper or cap.

FIG. 13B is a section view of the adaptor of FIG. 13A, as seen along line 13B-13B.

FIG. 13C is an enlarged view of a detailed portion 13C of the section view in FIG. 13B.

FIG. 13D is an enlarged view of a detailed portion 13D of the section view in FIG. 13B.

FIG. 14 is an isometric view of the additional alternative embodiment of the adaptor with a tethered removable stopper or cap.

FIG. 15A is a plan view of the front of the adaptor of FIG. 14 with the optional tethered removable stopper or cap.

FIG. 15B is a section view of the adaptor of FIG. 15A, as seen along line 15B-15B.

FIG. 15C is an enlarged view of a detailed portion 15C of the section view in FIG. 15B.

FIG. 15D is an enlarged view of a detailed portion 15D of the section view in FIG. 15B.

FIG. 16 is an isometric view of another additional alternative embodiment of the adaptor.

FIG. 17A is a plan view of the front of the adaptor of FIG. 16 with an optional tethered removable stopper or cap.

FIG. 17B is a section view of the adaptor of FIG. 17A, as seen along line 17B-17B.

FIG. 17C is an enlarged view of a detailed portion 17C of the section view in FIG. 17B.

FIG. 18A is a plan view of the front of another embodiment of the adaptor of FIG. 16 with an optional tethered removable stopper or cap.

FIG. 18B is a section view of the adaptor of FIG. 18A, as seen along line 18B-18B.

FIG. 18C is an enlarged view of a detailed portion 18C of the section view in FIG. 18B.

FIG. 19 is an isometric view of another additional alternative embodiment of the adaptor.

FIG. 20A is a plan view of the front of the adaptor of FIG. 19 with an optional tethered removable stopper or cap.

FIG. 20B is a section view of the adaptor of FIG. 20A, as seen along line 20B-20B.

FIG. 21A is a plan view of the front of the adaptor of FIG. 19 with an optional tethered removable stopper or cap.

FIG. 21B is a section view of the adaptor of FIG. 21A, as seen along line 21B-21B.

### DETAILED DESCRIPTION

The present invention provides, inter alia, devices and systems for pulmonary delivery of one or more active agents as a fluid, preferably as aerosol or gas to a patient, concomitantly with administration of noninvasive or invasive ventilatory support.

Unless otherwise indicated the terminology used herein is for the purpose of describing particular embodiments only and is not intended to limit the scope of the present invention. It must be noted that as used herein and in the claims, the singular forms “a,” “and” and “the” include plural referents unless the context clearly dictates otherwise.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “active agent” as used herein refers to a substance or combination of substances or devices that can be used for therapeutic purposes (e.g., a drug), diagnostic purposes or prophylactic purposes via pulmonary delivery. For example, an active agent can be useful for diagnosing the presence or absence of a disease or a condition in a patient and/or for the treatment of a disease or condition in a patient. Certain “active agents” are substances or combinations of substances that are capable of exerting a biological effect when delivered by pulmonary routes. The bioactive agents can be neutral, positively or negatively charged. Exemplary agents include, for example, insulins, autocoids, antimicrobials, antipyretics, antiinflammatories, surfactants, antibodies, antifungals, antibacterials, analgesics, anorectics, antiarthritics, antispasmodics, antidepressants, antipsychotics, antiepileptics, antimalarials, antiprotozoals, anti-gout agents, tranquilizers, anxiolytics, narcotic antagonists, antiparkinsonisms, cholinergic agonists, antithyroid agents, antioxidants, antineoplastics, antivirals, appetite suppressants, antiemetics, anticholinergics, antihistaminics, antimigraines, bone modulating agents, bronchodilators and anti-asthma drugs, chelators, antidotes and antagonists, contrast media, corticosteroids, mucolytics, cough suppressants and nasal decongestants, lipid regulating drugs, general anesthetics, local anesthetics, muscle relaxants, nutritional agents, parasympathomimetics, prostaglandins, radio-pharmaceuticals, diuretics, antiarrhythmics, antiemetics, immunomodulators, hematopoietics, anticoagulants and thrombolytics, coronary, cerebral or peripheral vasodilators, hormones, contraceptives, diuretics, antihypertensives, cardiovascular agents such as cardiotonic agents, narcotics, vitamins, vaccines, medical gases such as, for example nitric oxide, helium, xenon, carbon monoxide, hydrogen sulfate, oxygen, anesthetic agents such as nitrous oxide and halogenated agents (e.g., halothane, enflurane, isoflurane, desflurane, and sevoflurane) and the like.

In one embodiment, the active agent employed is a high-dose therapeutic. Such high dose therapeutics would include antibiotics, such as amikacin, gentamicin, colistin, tobramycin, amphotericin B. Others would include mucolytic agents such as N-acetylcysteine, Nacystelyn, alginase, mercaptoethanol and the like. Antiviral agents such as ribavirin, gancyclovir, neuraminidase inhibitors and the like, diamidines such as pentamidine and the like, and proteins such as antibodies are also contemplated.

A preferred active agent is a substance or combination of substances that is used for pulmonary prophylactic or rescue therapy, such as a pulmonary surfactant (PS) or medical gas.

Natural PS lines the alveolar epithelium of mature mammalian lungs. Natural PS has been described as a “lipoprotein complex” because it contains both phospholipids and apoproteins that act in conjunction to modulate the surface tension at the lung air-liquid interface and stabilize the alveoli to prevent their collapse. Four proteins have been found to be associated with pulmonary surfactant, namely SP-A, SP-B, SP-C, and SP-D (Ma et al., Biophysical Journal 1998, 74:1899-1907). Specifically, SP-B appears to impart the full biophysical properties of pulmonary surfactant when associated with the appropriate lung lipids. An absence of SP-B is associated with respiratory failure at birth. SP-A, SP-B, SP-C, and SP-D are cationic peptides that can be derived from animal sources or synthetically. When an animal-derived surfactant is employed, the PS is often bovine or porcine derived.

For use herein, the term PS refers to both naturally occurring and synthetic pulmonary surfactant. Synthetic PS, as used herein, refers to both protein-free pulmonary surfactants and pulmonary surfactants comprising synthetic peptides or peptide mimetics of naturally occurring surfactant protein. Any PS currently in use, or hereafter developed for use in RDS and other pulmonary conditions, is suitable for use in the present invention. Exemplary PS products include, but are not limited to, lucinactant (Surfaxin®, Discovery Laboratories, Inc., Warrington, Pa.), poractant alfa (Curosurf®, Chiesi Farmaceutici SpA, Parma, Italy), beractant (Survanta®, Abbott Laboratories, Inc., Abbott Park, Ill.) and colfosceril palmitate (Exosurf®, GlaxoSmithKline, PLC, Middlesex, U.K.).

While the methods and systems of this invention contemplate use of active agents, such as pulmonary surfactant compositions, antibiotics, antivirals, mucolytic agents, as described above, the preferred active agent is a synthetic pulmonary surfactant. From a pharmacological point of view, the optimal exogenous PS to use in the treatment would be completely synthesized in the laboratory. In this regard, one mimetic of SP-B that has found to be useful is KL4, which is a 21 amino acid cationic peptide. Specifically the KL4 peptide enables rapid surface tension modulation and helps stabilize compressed phospholipid monolayers. KL4 is representative of a family of PS mimetic peptides which are described for example in U.S. Pat. Nos. 5,260,273 and 5,407,914. Preferably, the peptide is present within an aqueous dispersion of phospholipids and free fatty acids or fatty alcohols, e.g., DPPC (dipalmitoyl phosphatidylcholine) and POPG (palmitoyl-oleyl phosphatidylglycerol) and palmitic acid (PA). See, for example, U.S. Pat. No. 5,789,381.

As used herein, the term “aerosol” refers to liquid or solid particles that are suspended in a gas. Typically, the “aerosol” or “aerosolized agent” referred to herein contains one or more of the active agents, as referred to above. The aerosol can be in the form of a solution, suspension, emulsion, powder, solid, or semi-solid preparation. Although, not typically considered as aerosol, for the purposes of this disclosure, this term is used interchangeably with the term “fluids” and further includes liquids and gasified active agents or a medical gas without liquid or solid particles dispersed therein. Consequently, any conduits or parts described in association with the term “aerosol” should be interpreted in the above described manner as capable to be used with fluids.

The term “ventilation” or “respiratory ventilation” as used herein refers to mechanical or artificial support of a patient's breathing. The principles of mechanical ventilation are governed by the Equation of Motion, which states that the amount of pressure required to inflate the lungs depends upon resistance, compliance, tidal volume and inspiratory flow. The principles of mechanical ventilation are described in detail in Hess and Kacmarek, ESSENTIALS OF MECHANICAL VENTILATION, 2^{nd} Edition, McGraw-Hill Companies (2002). The overall goals of mechanical ventilation are to optimize gas exchange, patient work of breathing and patient comfort while minimizing ventilator-induced lung injury. Mechanical ventilation can be delivered via positive-pressure breaths or negative-pressure breaths. Additionally, the positive-pressure breaths can be delivered noninvasively or invasively.

Noninvasive mechanical ventilation (NIMV) generally refers to the use of a mask or nasal prongs to provide ventilatory support through a patient's nose and/or mouth. The most commonly used interfaces for noninvasive positive pressure ventilation are nasal prongs, nasopharyngeal tubes, masks, or oronasal masks. Desirable features of a mask for noninvasive ventilation include low dead space, transparent, lightweight, easy to secure, adequate seal with low facial pressure, disposable or easy to clean, nonirritating to the skin (non-allergenic) and inexpensive.

NIMV is distinguished from those invasive mechanical ventilatory techniques that bypass the patient's upper airway with an artificial airway (endotracheal tube, laryngeal mask airway or tracheostomy tube). NIMV can be provided by either bi-level pressure support (so called “BI-PAP”) or continuous positive airway pressure (CPAP). Bi-level support provides an inspiratory positive airway pressure for ventilatory assistance and lung recruitment, and an expiratory positive airway pressure to help recruit lung volume and, more importantly, to maintain adequate lung expansion. Continuous positive airway pressure provides a single level of airway pressure, which is maintained above atmospheric pressure throughout the respiratory cycle. For a further review of invasive and noninvasive mechanical ventilation, see Cheifetz, I. M., Respiratory Care, 2003, 48:442-453.

The employment of mechanical ventilation, whether invasive or non-invasive, involves the use of various respiratory gases, as would be appreciated by the skilled artisan. Respiratory gases pulmonary respiratory therapy are sometimes referred to herein as “CPAP gas,” “ventilation gas,” “ventilation air,” or simply “air.” However, those terms are intended to include any type of gas normally used for respiratory therapy. The terms “channel” and “chamber” are used interchangeably in this disclosure and are not intended to be limited to any particular shape or form.

The term “a delivery means” when used together with ventilation gas refer to a conduit or a network of conduits containing (if needed) various devices (pressure valves, sensors, etc.) necessary to enable delivery of ventilation gas, preferably pressurized ventilation gas, to and from the adaptor. The type of conduits, their geometry and materials they are made of are not limited to any specifics. A person skilled in the art should be able to select appropriate conduits and devices based on the teaching disclosed herein and knowledge available in the art.

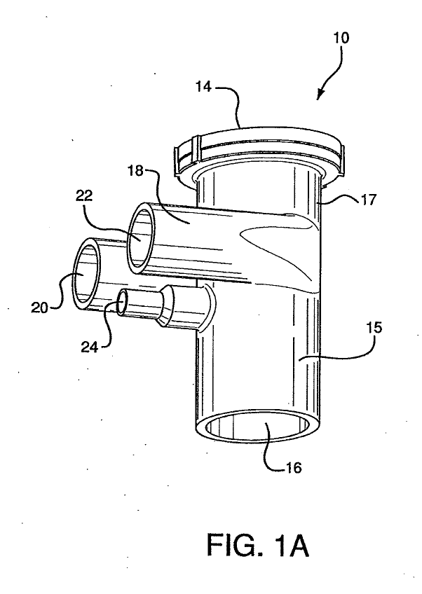

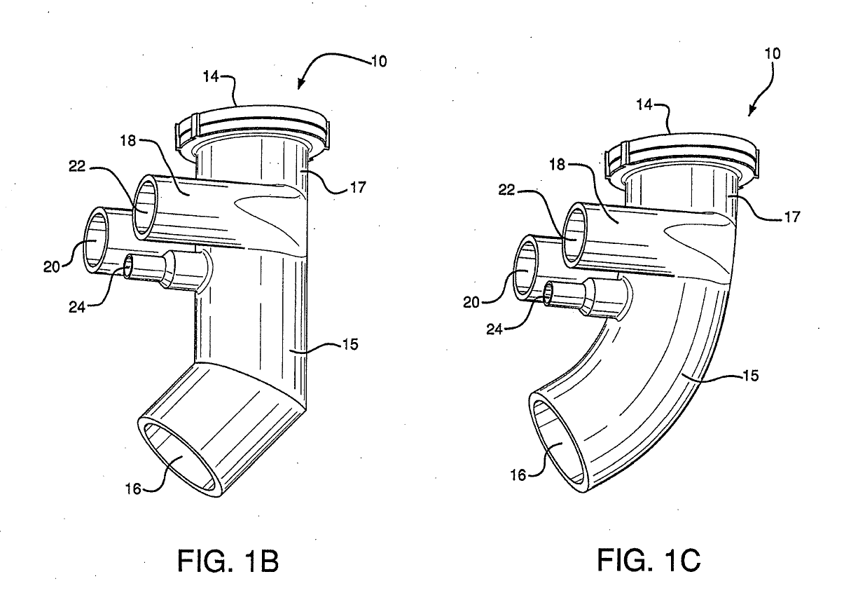

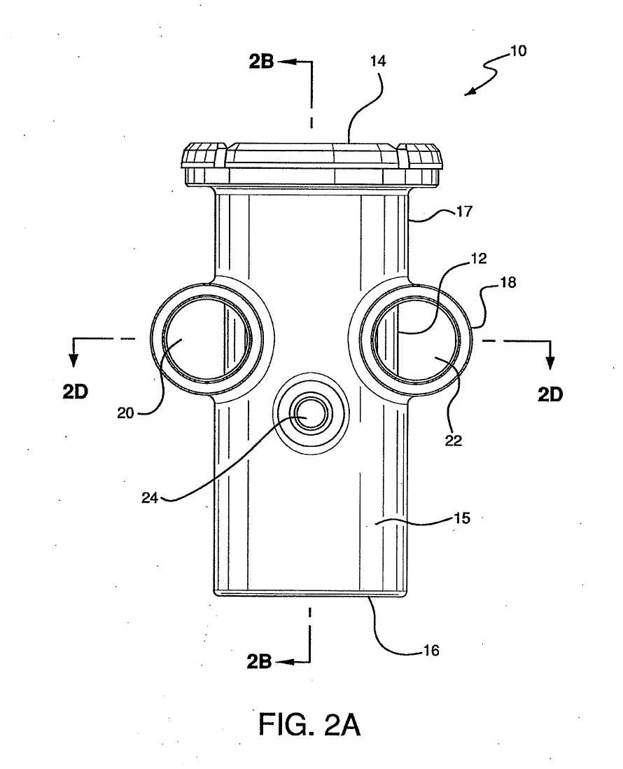

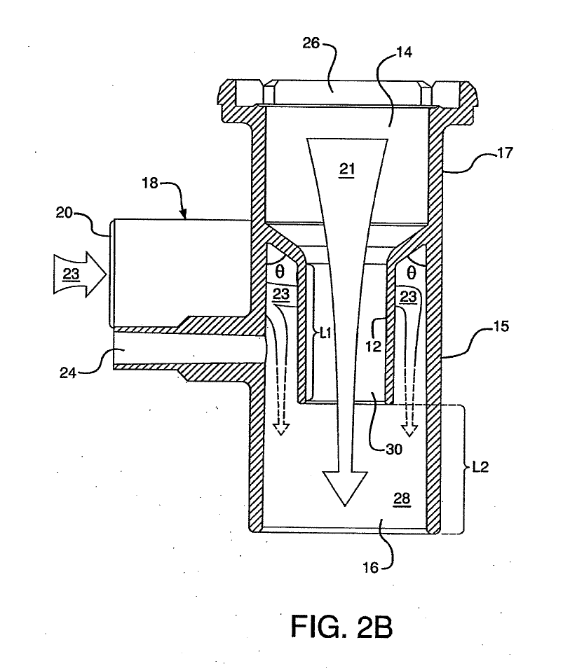

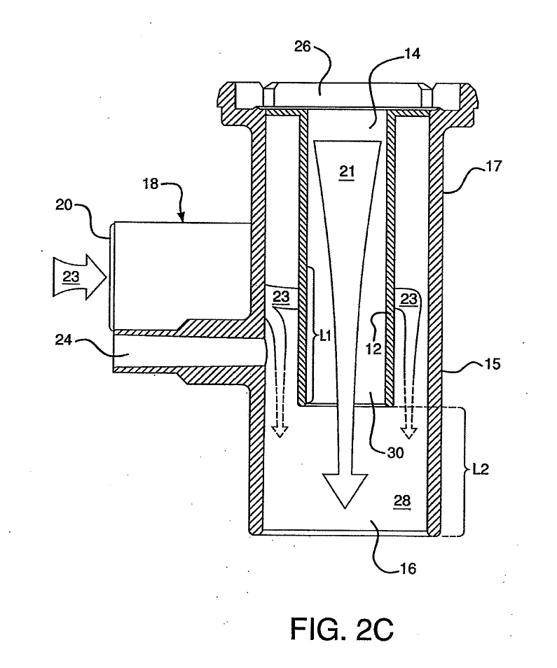

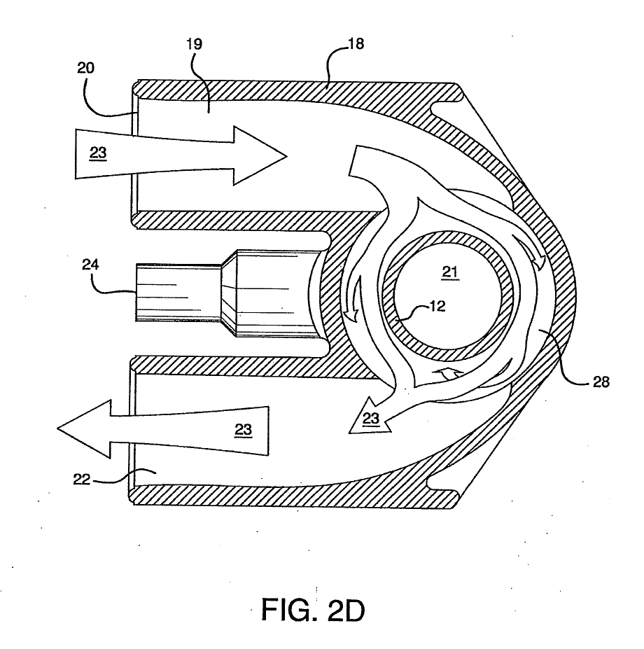

Turning now to the drawings, FIG. 1A shows an embodiment of the ventilation circuit adaptor 10 including a body 15, an aerosol flow chamber 17 and a ventilation gas flow chamber 18. The aerosol flow chamber 17 comprises an aerosol inlet port 14 with an optional valve (not visible) and a patient interface port 16. As shown in FIG. 2B, aerosol is passed from an aerosol generator (not shown) directly or indirectly (e.g., via tubing) through the aerosol inlet port 14 into the aerosol flow channel 12 and out of the aerosol flow channel 12 to the patient via the aerosol outlet port 30 to and through the patient interface port 16. The patient interface port 16 is connected directly or indirectly (e.g., via tubing) to a patient interface, such as an endotracheal tube, a mask or nasal prongs (not shown). As shown in FIG. 1A, the ventilation gas flow chamber 18 comprises ventilation gas inlet and outlet ports 20 and 22, respectively. It is understood that the inlet and the outlet can be switched such that the inlet can become an outlet and the outlet can become the inlet. In this embodiment, the ventilation gas flow chamber 18 is joined with the aerosol flow chamber 17 to facilitate flow of the aerosol without dilution with ventilation gas or with a minimum dilution as shown more fully in FIGS. 2A-4. The body 15 further comprises an optional pressure sensor port 24. While the main body of the adaptor 10 is preferably roughly cylindrical along its length, it will be appreciated by one of skill in the art that the body of the adaptor 10 may utilize any cross-sectional shape.

FIGS. 1B and 1C illustrate alternative embodiments of the adaptor shown in FIG. 1A. FIG. 1B shows an angled configuration; FIG. 1C shows a curved configuration.

FIGS. 2A-2D illustrate the embodiment of the adaptor shown in FIG. 1A in more detail. As seen in FIG. 2A, the ventilation gas flow chamber 18 is joined with an aerosol flow chamber 17 to form a combined body 15 which houses a chamber 28 (as illustrated in FIGS. 2B, 2C, and 4). The aerosol flow channel 12 is nested within the chamber 28. As shown in FIG. 2B, the aerosol 21 is introduced into the aerosol flow channel 12 via aerosol inlet port 14, through valve 26. The aerosol 21 flows through the aerosol flow channel 12 to and through the aerosol outlet port 30, then to and through the patient interface port 16. The length L1 of the aerosol flow channel 12 is sufficient to extend beyond the ventilation gas flow chamber 18, but is recessed within the chamber 28 by a length L2 to minimize resistance arising from the patient's exhalations. The inventors have discovered that selecting the proper value for L1 has a direct impact on the volume of ventilation gas which reaches the patient interface port. Ventilation gas 23 is introduced through gas inlet port 20 into a ventilation gas flow channel 19 (shown in FIG. 2D) and follows a flow path that partially encircles the aerosol flow channel 12, but may be pulled toward the patient interface port 16 under certain circumstances (e.g., when aerosol flow is not being generated or when the aerosol flow rate is less than the patient's inspiratory flow (PIF) as indicated by “broken lines” in FIGS. 2B and 2C). As shown in FIG. 2B, the aerosol flow channel 12 occupies the entire volume of the aerosol flow chamber 17 at the portion near the aerosol inlet port 14 and above the ventilation gas flow chamber 18, then narrows between the ventilation gas flow chamber 18 and the aerosol outlet port 30 and thus creating a separation barrier between the aerosol flow and the ventilator flow, to enable the ventilation gas flow chamber 18 to at least partially encircle the aerosol flow channel 12. The separation barrier between the aerosol flow and the ventilator flow has a predetermined length L1. The inventors have discovered that introducing the aerosol to the chamber 28 at a point below the ventilation gas flow channel prevents high ventilatory flow rates from diluting the aerosol or at least decreases the aerosol dilution effect, thus allowing more of the aerosol to reach the patient interface. In order to maximize aerosol inhaled dose and decrease aerosol losses, the aerosol flow is selected to match the PIF. Nevertheless, ventilator flow rates are always significantly higher than PIF. Thus, by separation of aerosol flow from higher ventilator flows, aerosol dilution, which occurs whenever aerosol flow is introduced directly to the ventilatory flow path, can be avoided or minimized. Using the adaptor of the invention, the amount of the ventilation gas delivered to the patient can be regulated by selecting the length of the aerosol flow channel and/or regulating the pressure created by an increased demand for air which is not matched by the aerosol flow (e.g., when PIE is higher than the aerosol flow rate).

As shown in FIG. 2B, the aerosol flow channel 12 forms a funnel-like shape. This arrangement minimizes corners, and thus helps to prevent the accumulation of deposits within the adaptor. In an alternative embodiment shown in FIG. 2C, the aerosol flow channel 12 is substantially the same diameter throughout its length, and is not configured as a funnel. In either embodiment, the aerosol flow channel 12 is sufficiently narrower than the chamber 28 to allow for flow of ventilation gas 23 around the aerosol flow channel 12.

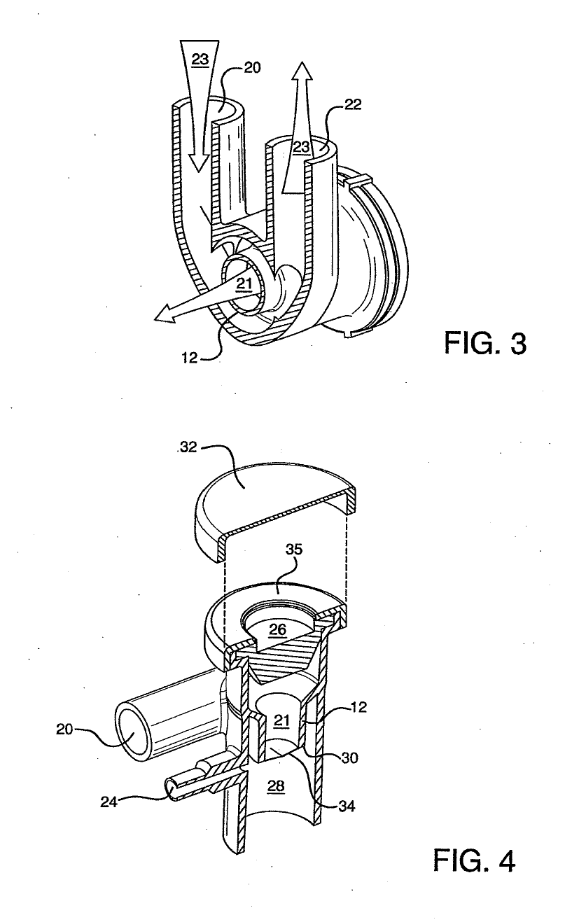

FIGS. 2D and 3 show the arrangement of the ventilation gas inlet and outlet ports 20/22 and the optional pressure sensor port 24, and the flow of ventilation gas around the aerosol flow channel 12. Ventilation gas flows into the ventilation gas flow channel 19 through port 20 and out through port 22, with a portion being pulled toward the patient interface port 16 through the chamber 28, substantially parallel to the aerosol flow path 21, under certain circumstances (e.g., when aerosol flow is not being generated or when the aerosol flow rate is less than the patient's inspiratory flow).

FIG. 4 illustrates the arrangement of the aerosol inlet port at the top of the adaptor. A removable cap 32 is shown. The cap 32 may be utilized when the aerosol generator is not being used, and removed when the adaptor is connected to an aerosol generator. The aerosol flows through valve 26 into the aerosol flow channel 12. The valve 26 is preferably a slit or cross-slit valve of the type known in the art. When an aerosol generator is attached to the adaptor, the valve 26 is forced into an open position. When the aerosol generator is removed, the valve 26 closes. The adaptor 10 may further comprise a one-way valve 34 at the aerosol outlet port 30, to reduce or prevent any reverse aerosol flows that might occur during excessive expirations. A security lock 35 is used to prevent dislocation of valve 26.

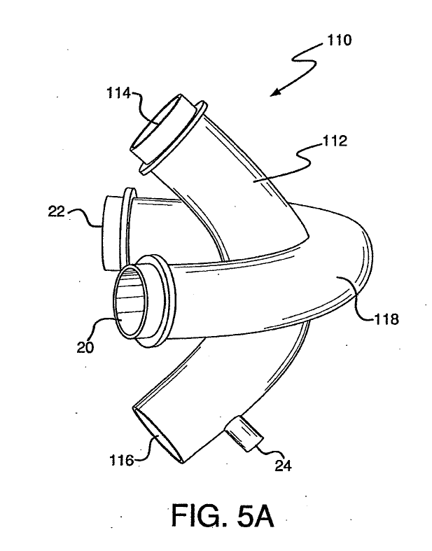

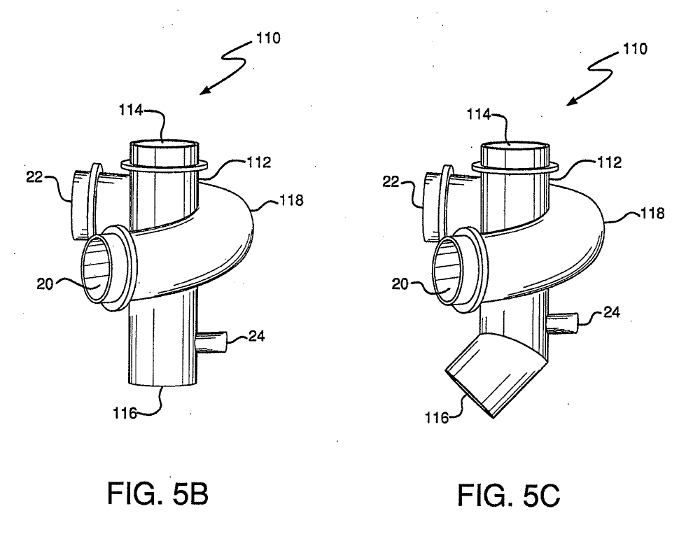

FIG. 5A shows another embodiment of the ventilation circuit adaptor 110, which includes an aerosol flow channel 112 and a ventilation gas flow channel 118. Similarly to the adaptor shown in FIGS. 1A-4, the aerosol flow channel 112 comprises an aerosol inlet port 114 with an optional valve (not visible) and a patient interface port 116. The ventilation gas flow channel 118 comprises ventilation gas inlet and outlet ports 20 and 22, respectively. In this embodiment, the ventilation gas flow channel is not adapted to form a chamber through which passes the aerosol flow channel. Instead, the aerosol flow channel 112 and the ventilation gas flow channel 118 are formed as substantially separated tubes, in fluid communication by means of an aperture 36 (shown in FIG. 7). In the embodiment shown, the optional pressure sensor port 24 is placed in the aerosol flow channel 112, near the patient interface. While the two flow channels are roughly tubular in shape, it will be appreciated by one of skill in the art that either or both channels may be of any cross-sectional dimension.

FIGS. 5B and 5C illustrate alternative embodiments of the adaptor shown in FIG. 5A. FIG. 5B shows a straight configuration for the aerosol flow channel 112; FIG. 5C shows an angled configuration for the aerosol flow channel 112.

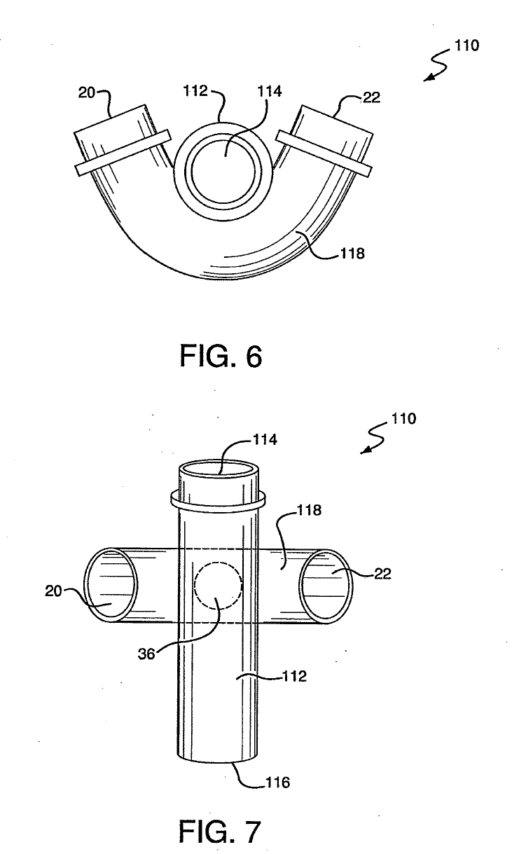

FIG. 6 and FIG. 7 illustrate the embodiment of the adaptor shown in FIG. 5B viewed from different angles. As seen in the top view of FIG. 6 and the front view of FIG. 7, the ventilation gas flow channel 118 is substantially separated from the aerosol flow channel 112, and is in fluid communication therewith by means of an aperture 36. Aerosol is introduced into the aerosol flow channel 112 via aerosol inlet port 114, through optional valve 126 (not shown). The aerosol flows through the aerosol flow channel 112 to and through the patient interface port 116. Ventilation gas is introduced through gas inlet port 20 and follows a flow path that partially encircles the aerosol flow channel and exits at gas outlet port 22, but may move through the aperture 36 into the aerosol flow channel 112, toward the patient interface port 116 under certain circumstances (e.g., when aerosol flow is not being generated or when the aerosol flow rate is less than the patient's inspiratory flow).

Although Applicants' adaptors of certain dimensions may be manufactured as one piece, manufacturing problems have been encountered for adaptors having some larger dimensions or when addressing the need for reducing the overall size of the adaptor by using different diameters of an aerosol chamber inlet port and a patient interface port. For example, current tooling constraints prevent one-piece manufacture of Applicants' adaptor having an aerosol channel inlet port with a 22 mm internal diameter and a patient interface port with a 15 mm internal diameter (“larger adaptor”). Whereas a smaller adaptor having a 15 mm inner diameter for both the aerosol channel inlet port and the patient interface port allowed for insertion and ejection of tooling pins while forming and releasing of an adaptor as one piece during the molding process, that was not possible for the “larger adaptor” with current tooling.

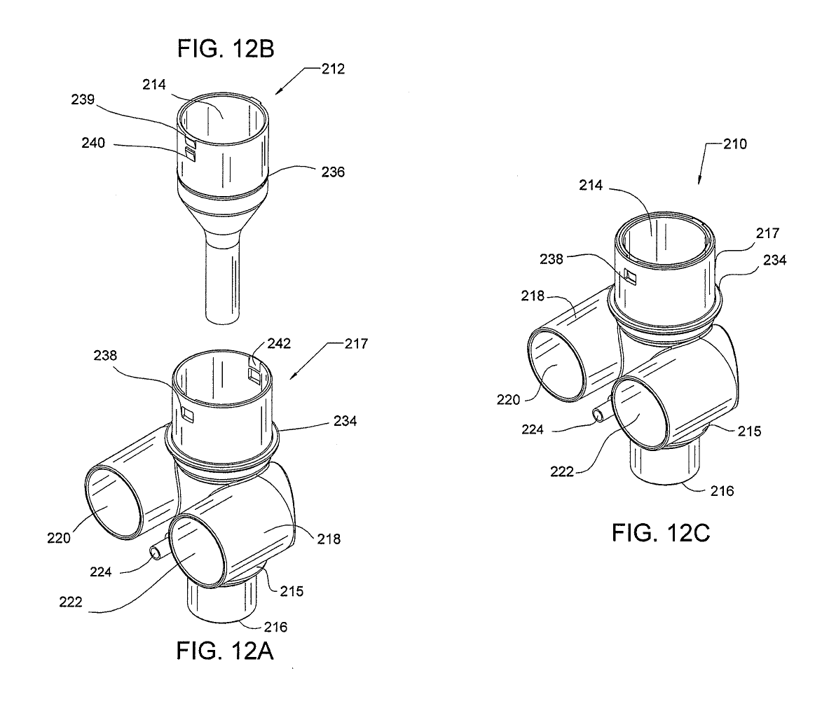

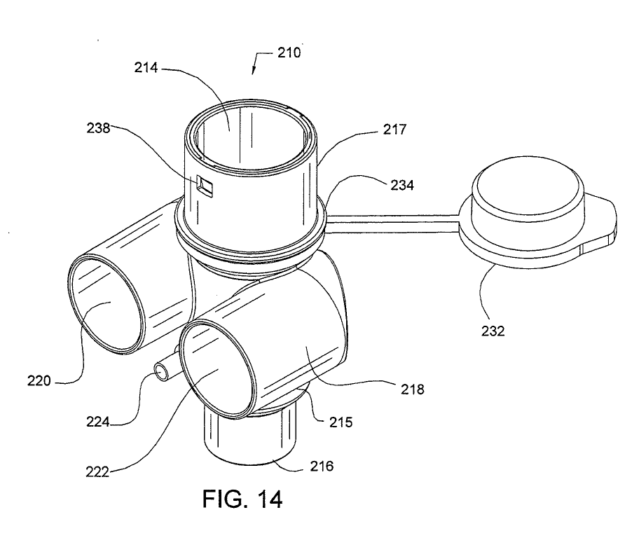

To address such manufacturing problems and related issues, additional alternative embodiments of Applicants' ventilation circuit adaptor were developed together with methods for assembling such adaptors. FIGS. 12A through 21B illustrate additional alternative embodiments of Applicants' ventilation circuit adaptor.

FIG. 12C shows an embodiment of a ventilation circuit adaptor 210 assembled from the two components shown in FIGS. 12A and 12B, an aerosol flow chamber 217 and a funnel-shaped aerosol flow channel 212.

The first component, the aerosol flow chamber 217 shown in FIG. 12A, has a body 215, a ventilation gas flow chamber 218, an aerosol chamber inlet port with an optional valve (not visible), and a patient interface port 216. The patient interface port 216 is connected directly or indirectly (e.g., via tubing) to a patient interface, such as an endotracheal tube, a mask or nasal prongs (not shown). The ventilation gas flow chamber 218 has ventilation gas inlet and outlet ports 220 and 222, respectively. The inlet and the outlet can be switched such that the inlet port can become the outlet port and the outlet port can become the inlet port. The body 215 may include an optional pressure sensor port 224. While the body 215 of the ventilation circuit adaptor 210, as illustrated, is generally cylindrical along its length, persons skilled in the art will appreciate that the body 215 and the ventilation circuit adaptor 210 may have other shapes and other cross-sectional areas.

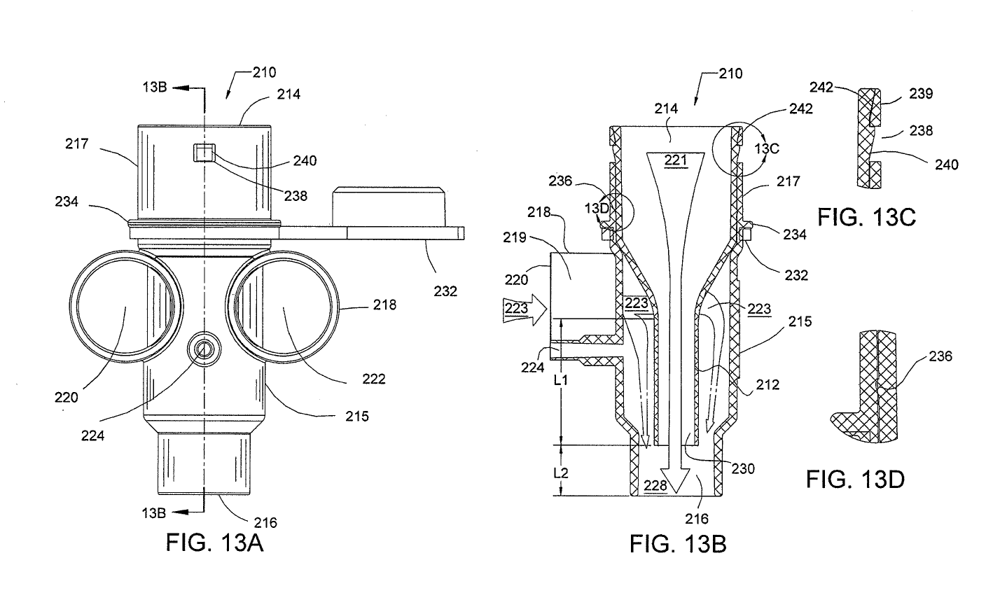

The other component of this embodiment of the ventilation circuit adaptor 210 is shown in FIG. 12B—a funnel-shaped aerosol flow channel 212 which is adapted to be inserted into, and fixedly positioned in, the aerosol flow chamber 217. When inserted, as shown in FIG. 12C, the longitudinal axis of the funnel-shaped aerosol flow channel 212 is coaxial with the longitudinal axis of the aerosol flow chamber 217. In this position the funnel-shaped aerosol flow channel 212 is nested within the chamber 228 as shown in FIG. 13B, similar to the embodiment illustrated in FIG. 2B.

As shown in FIG. 13B, the aerosol 221, is introduced into the funnel-shaped aerosol flow channel 212 via aerosol channel inlet port 214. The aerosol 221 flows through the funnel-shaped aerosol flow channel 212 to and through the aerosol outlet port 230, then to and through the patient interface port 216. The length L1 of the funnel-shaped aerosol flow channel 212 is sufficient to extend beyond the ventilation gas flow chamber 218, but is recessed within the chamber 228 by length L2 to minimize resistance arising from the patient's exhalations. (As previously discussed with respect to the embodiments of the adaptors shown in FIGS. 2B and 2C, selecting the proper value for L1 has a direct impact on the volume of ventilation gas which reaches the patient interface port.)

As illustrated in FIG. 13B, the ventilation gas 223 is introduced through gas inlet port 220 into a ventilation gas flow channel 219 and follows a flow path that partially encircles the aerosol flow channel 212, but may be pulled toward the patient interface port 216 under certain circumstances (e.g., when aerosol flow is not being generated or when the aerosol flowrate is less than the patient's inspiratory flow (PIF) (as indicated by “broken lines” in FIG. 13B).

As shown in FIG. 13B, the funnel-shaped aerosol flow channel 212 occupies the entire volume of the aerosol flow chamber 217 at the portion near the aerosol channel inlet port 214 and above the ventilation gas flow chamber 218, then narrows between the ventilation gas flow chamber 218 and the aerosol outlet port 230, thus creating a separation barrier between the aerosol flow and the ventilator flow, to enable the ventilation gas flow chamber 218 to at least partially encircle the aerosol flow channel 212. The separation barrier between the aerosol flow and the ventilator flow has a predetermined length L1.

As shown in FIGS. 12B and 13B, the aerosol flow channel 212 has a funnel-like shape, similar to that in the embodiment illustrated in FIG. 2B. The aerosol flow channel 212 is sufficiently narrower than the chamber 228 to allow for flow of ventilation gas 223 around the aerosol flow channel 212. The funnel-shaped aerosol flow channel 212 has smooth and contoured radii to prevent flow turbulence and provide safety.

FIGS. 13A, 14, and 15A illustrate the optional use of a tethered removable cap or plug 232. The removable cap or plug may be used when an aerosol generator is not being used, and may be removed when the ventilation circuit adaptor 210 is connected to an aerosol generator (not shown). The outer diameter of the body 215 above the ventilation gas inlet and outlet ports 220 and 222 includes a retaining ring 234 adapted to limit the movement of the tethered removable plug or cap 232.

As illustrated in FIGS. 13B and 13D, the funnel-shaped aerosol flow channel 212 has a positive interference seal 236 which is integral to, and completed as part of, the molding process. This positive interference seal 236 prevents gases from leaking up between the inner wall of the aerosol flow chamber 217 and the outer wall of the funnel-shaped aerosol flow channel 212. Use of the positive interference seal 236 avoids the need for elastomeric “O” rings incorporated into and between those components. In one embodiment, as illustrated in FIGS. 13A and 13B, a retaining ring 234 is positioned near the location of the positive interference seal 236 and adds rigidity and strength around the positive interference seal 236, thereby supporting the positive interference seal 236.

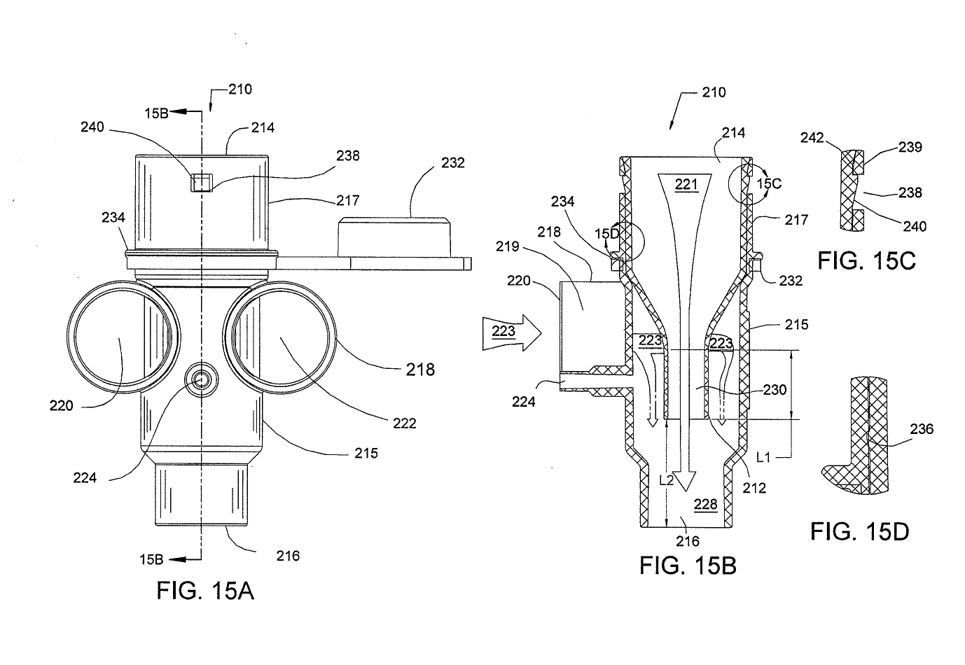

One embodiment uses a molded polycarbonate positive interference seal 236 that includes a ridge protruding from the outer wall of the funnel-shaped aerosol flow channel 212, as illustrated in FIGS. 13D and 15D. This type of seal has some advantages over elastomeric “O” rings because use of such a positive interference seal 236 reduces the potential hazard of elastomeric particle shedding, contamination areas, and assembly failure by reducing the number of parts.

There is a one degree draft between the outer and inner components of the molded seal assembly (i.e., between the aerosol flow chamber 217 and the funnel-shaped aerosol flow channel 212). This draft facilitates the insertion of the inner funnel-shaped aerosol flow channel 212 into position inside the aerosol flow chamber 217. When the funnel-shaped aerosol flow channel 212 is in position there is an interference fit of less than about 0.010 inches between those inner and outer components of the ventilation circuit adaptor 210. This interference fit between the positive interference seal 236 and the inner wall of the aerosol flow chamber 217 creates a positive fit where the positive interference seal 236 meets the inner wall of the aerosol flow chamber 217 and prevents leakage of gases up through the region.

As illustrated in FIGS. 12A, 12B, and 12C, the aerosol flow chamber 217 has two apertures 238 that capture the snap-in catches 240 located on the outside diameter of the funnel-shaped aerosol flow channel 212. When mated, these two components (i.e., the snap-in catch 240 and the aperture 238) lock in place to secure the two components (212 and 217) of the ventilation circuit adaptor 210. Persons skilled in the art will recognize that this assembly alignment fixture is but one way to achieve this result and that other assembly alignment fixtures could be used as well.

In the embodiment illustrated in FIGS. 12A, 12B, and 12C, a snap-in catch 240 is used in two places located about 180° from each other around the circumference of the two components (212 and 217) illustrated in FIGS. 12A and 12B. This feature prevents the funnel-shaped aerosol flow channel 212 from rotating within the aerosol flow chamber 217, as well as secures the placement of the ridge shown in FIGS. 13D and 15D that creates the positive interference seal 236.

As shown in FIG. 13C, above the apertures 238 are alignment receptacles 242 which locate the snap-in catches 240 and vertically guide the snap-in catches 240 into the apertures 238. When the snap-in catches 240 are placed in the apertures 238, the funnel-shaped aerosol flow channel 212 is positioned inside the aerosol flow chamber 217. In addition, plug 239, shown in FIG. 12B, seats within alignment receptacle 242, shown in FIG. 12A, when the snap-in catch 240 is positioned in aperture 238. This helps to prevent rotation of the snap-in catch 240 out of the apertures 238.

The embodiment of the ventilation circuit adaptor 210 illustrated in FIGS. 15A-15D is similar to the embodiment of the ventilation circuit adaptor 210 shown in FIGS. 13B-13D. The differences between the two embodiments are the differences in the lengths of L1 and L2 which can be seen by comparing FIG. 15B to FIG. 13B.

In one example where this embodiment of the ventilation circuit adaptor 210 may be used, the aerosol channel inlet port 214 may have a 22 mm internal diameter and the patient interface port 216 may have a 15 mm internal diameter. These internal diameters (22 mm and 15 mm) in this example may be selected to fit existing endotracheal tube adaptors to facilitate connection of the ventilation circuit adaptor 210 to the patient interface. (Whereas an aerosol channel inlet port 214 with a 15 mm internal diameter may be suitable on a ventilation circuit adaptor 210 for an infant, a 22 mm internal diameter for the aerosol channel inlet port 214 may be suitable for an adult.)

The patient interface end of the aerosol flow chamber 217 transitions from a 22 mm internal diameter to a 15 mm internal diameter at a distance L2 from the bottom of the body 215 and is tapered allowing sufficient support to securely hold the connector on the endotracheal tube (not shown) in place.

The funnel-shaped aerosol flow channel 212 and the aerosol flow chamber 217 components for the ventilation circuit adaptor 210 may be assembled with either an arbor press or semi-automated pressurized equipment. The funnel-shaped aerosol flow channel 212 needs to have a press or force applied in a true/plumb vertical direction so that the positive interference seal 236 sits square and evenly on all surfaces within the aerosol flow chamber 217 as the funnel-shaped aerosol flow channel 212 moves into position. This assembly then gets pressed with a force sufficient to seal the ventilation circuit adaptor 210 as one unit. When fully assembled and sealed, the ventilation circuit adaptor 210 is tested to assure that all went well in assembly and that there is no leak at the positive interference seal 236.

The outside surface of the aerosol flow chamber 217 may have a raised arrow molded into the outside surface to provide a visual indication of the directional flow of the aerosol toward the patient interface.

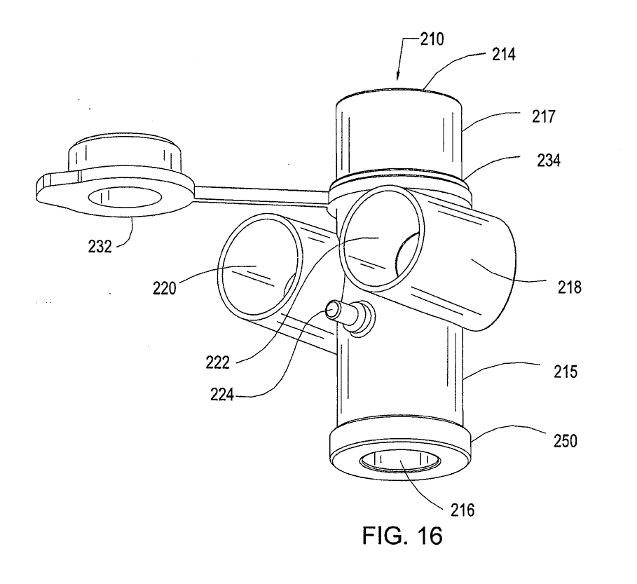

Variations of another alternative embodiment of the ventilation circuit adaptor 210 are shown in FIGS. 16-21B. This alternative embodiment is similar to the embodiment illustrated in FIG. 2B and previously described with reference to FIG. 2B. However, as shown in FIGS. 16-21B, this alternative embodiment includes a reducer 250 adjacent to the patient interface port 216. The inner diameter of the reducer 250 is smaller than the inner diameter of the patient interface port 216. The aerosol 221 flows through the aerosol flow channel 212 to and through the aerosol outlet port 230, then to and through the patient interface port 216, and then to and through the reducer 250.

As shown in FIGS. 16-21B, there are a number of variations of this alternative embodiment, each of which varies primarily in the way that the reducer 250 is connected to the bottom surface of the patient interface end of the body 215. In some variations, ultrasonic welding is used to attach the reducer 250 to the inner wall of the body 215. In other variations, the means for attaching the reducer 250 to the inner wall of the body 215 is laser welding. In another variation, the reducer 250, in the form of a flanged silicone bushing, is press fit into and against the inner wall of the body 215 at the patient interface end with a feature on the outer wall of the body 215 to further secure the bushing to the body 215. In all of the variations, an alternative means for connecting the reducer 250 to the bottom surface of the patient interface end of the body 215 is gluing. The variations illustrated in FIGS. 16 through 21B are discussed further below.

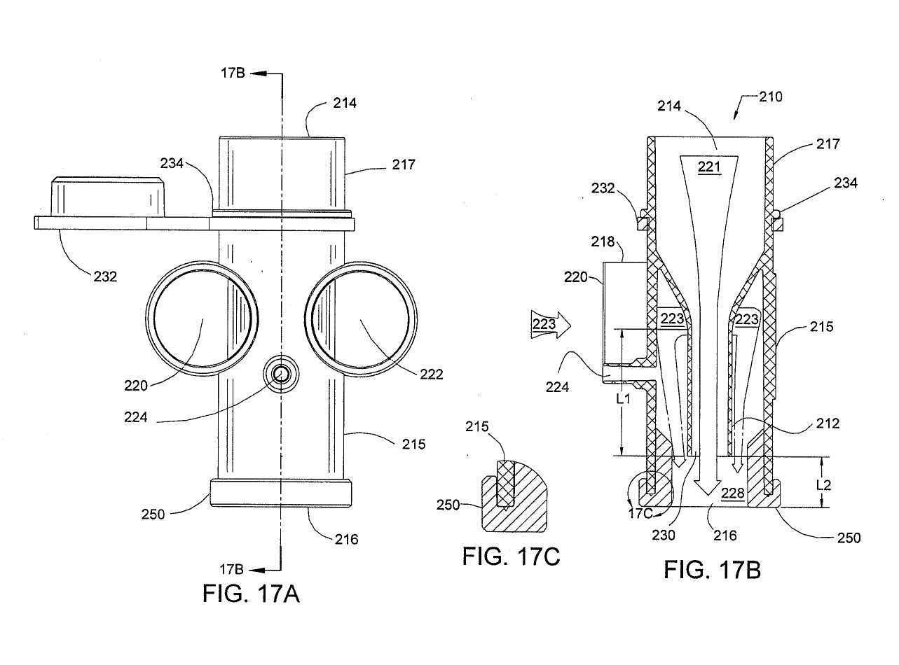

FIGS. 16, 17A-17B, and 18A-18C illustrate variations where ultrasonic welding is used to attach the reducer 250 to the inner wall of the body 215. The reducer 250 securely holds in place the connector on the patient interface endotracheal tube (not shown). As shown is FIG. 17C illustrating the detail at the bottom of FIG. 17B, the reducer 250 has a flash trap or cavity extending central to and around the bottom circumference of the reducer 250. This trap is where an energy director, which is positioned around the bottom surface of the body 215, seats for welding. The joint at this intersection directs the flow of energy, using high-frequency mechanical vibration, creating frictional heat and melding like thermoplastic polymers together. The joint and process together create a strong bond and a positive seal prohibiting escape of aerosol or gases.

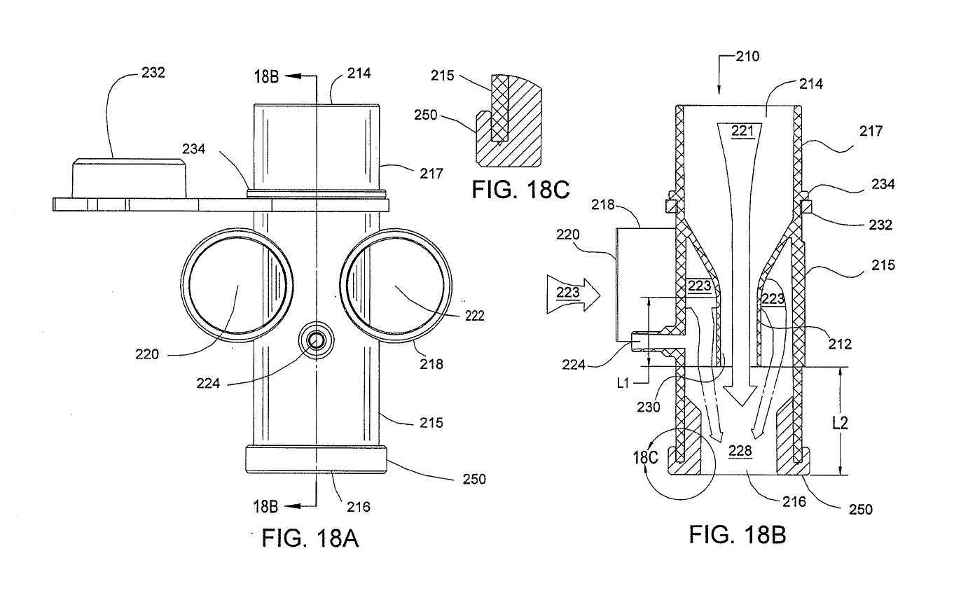

The embodiment of the ventilation circuit adaptor 210 illustrated in FIGS. 18A-18C is similar to the embodiment of the ventilation circuit adaptor 210 shown in FIGS. 17A-17B. The differences between the two embodiments are the differences in the length of L1 and L2 which can be seen by comparing FIG. 17B to FIG. 18B.

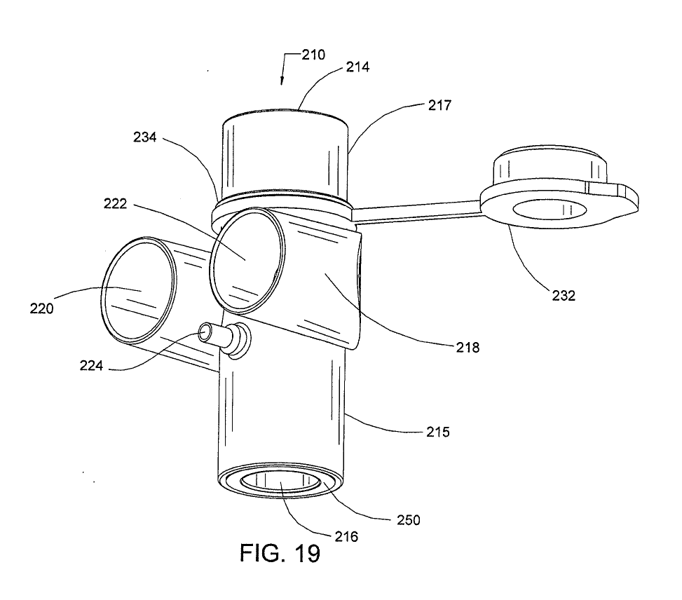

In FIGS. 19-21B, the reducer 250 is connected to the inner wall of the body 215 by laser welding. No flash trap is required in this embodiment.

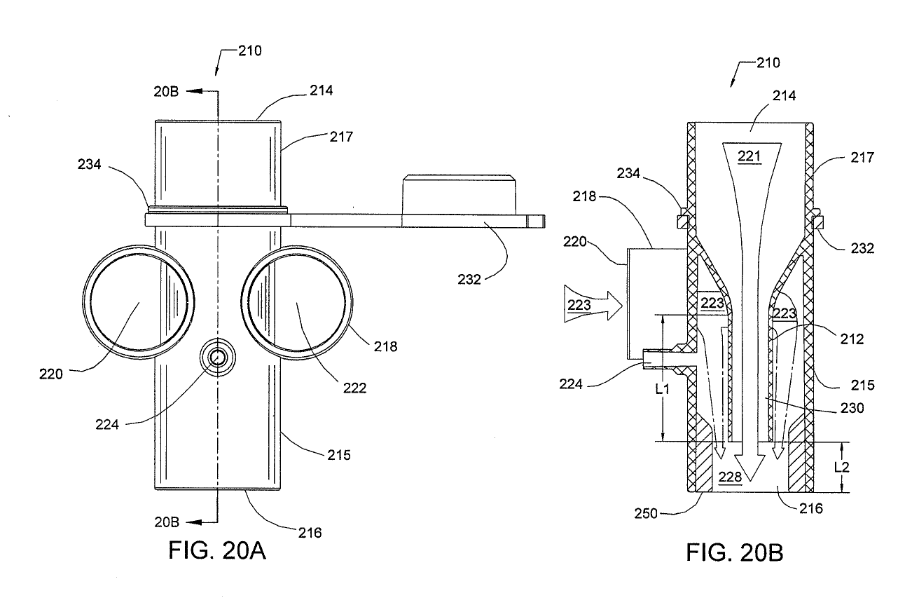

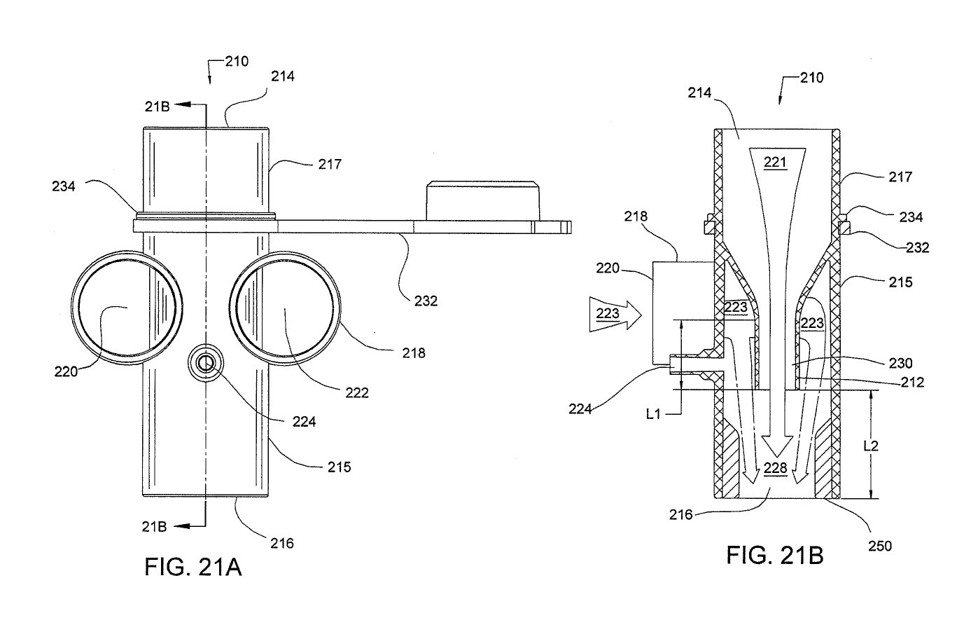

The embodiment of the ventilation circuit adaptor 210 illustrated in FIGS. 20A and 20B is similar to the embodiment of the ventilation circuit adaptor 210 shown in FIGS. 21A and 21B. The differences between the two embodiments are the differences in the lengths of L1 and L2, which can be seen by comparing FIG. 20B to FIG. 21B.

All surfaces of the reducer 250 and the body 215 have smooth and contoured radii for reduction of turbulence and safety. The fit of the body 215 and the reducer 250 when assembled is designed for optimal weld penetration and flash prevention.

Although the alternative embodiments of the ventilation circuit adaptor 210 illustrated in FIGS. 16-21B and discussed herein have a funnel-shaped aerosol flow channel 212, additional alternative embodiments may have an aerosol flow channel 212 that is substantially the same diameter throughout its length, and is not configured as a funnel. In such embodiments, the funnel-shaped aerosol flow channel 212 in FIGS. 16-21B would be replaced with an aerosol flow channel similar to the aerosol flow channel 12 shown in FIG. 2C.

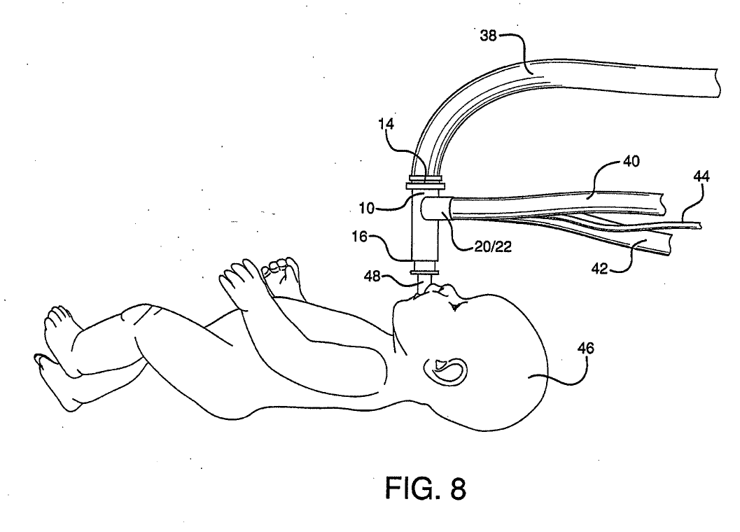

FIG. 8 depicts the arrangement of the adaptor 10 and various ventilatory and aerosol tubes of a system of the invention, as it may be used in a neonatal setting. It is understood that the adaptor can be used in any setting or with any apparatus suitable for pulmonary aerosol delivery. Tube 38 from the aerosol generator (generator not shown) is attached to the aerosol inlet port 14 of the adaptor 10. Ventilation gas inlet port 20 and outlet port 22 are affixed, respectively to tubes 40 and 42, which form the ventilatory circuit that includes the positive pressure generator (not shown). The pressure sensor port 24 (not shown) is attached via tubing 44 to a pressure sensor (pressure sensor not shown). The patient 46 is administered respiratory therapy through a patient interface, such as, for example, an endotracheal tube 48 which is affixed to the patient interface port 16.

The ventilation circuit adaptor of the present invention may be formed of, for example, polycarbonate or any other suitable material; however, materials such as molded plastic and the like, of a type used for tubing connectors in typical ventilatory circuits, are particularly suitable. The material utilized should be amenable to sterilization by one or more standard means. In certain embodiments, the adaptor is made of disposable materials. In certain embodiments, the adaptor is made of materials capable of withstanding temperatures and pressures suitable for sterilizing.

The adaptor may be of any size or shape within the functional parameters set forth herein. In a preferred embodiment, the adaptor is of a size and shape that enables its use with standard tubing and equipment used in mechanical ventilation circuits. This is of particular advantage over certain previously disclosed connectors (e.g., U.S. patent publication 2006/0120968 to Niven et al.), wherein the size of the chamber accounts for significant ventilation dead space, minimizing its effective use in invasive mechanical ventilation applications or other connectors (e.g., U.S. Pat. No. 7,201,167 to Fink et al.), wherein the aerosol is diluted with the ventilation gas. In particular embodiments, the adaptor is designed to replace the typical “Y” or “T” connector used in ventilatory circuits, and its size is such that no additional ventilation dead space is introduced into the ventilatory circuit. However, custom sizes and shapes may easily be fabricated to accommodate custom devices or equipment, as needed.

The ventilation circuit adaptor can comprise one or more optional features, either singly or in combination. These include: (1) one or more ports for attaching monitoring equipment, such as a pressure sensor; (2) a valve at the aerosol inlet port; (3) a removable cap for the aerosol inlet port; (4) a one-way valve at the aerosol outlet port; and (5) a temperature probe.

The port(s) for attaching monitoring equipment can be placed in various positions on the adaptor, as dictated by use with standard or custom equipment and in keeping with the intended function of the port. For instance, a pressure sensor port should be positioned on the adaptor such that ventilation and/or aerosol flow pressure can be accurately measured.

The valve at the aerosol inlet port is a particularly useful optional feature of the adaptor. Particularly suitable valves include slit or cross-slit valves. The valve is forced into an open position by attachment of an aerosol generator tube or the aerosol generator itself, and returns to a closed position when the aerosol generator tube is disconnected. As would be readily appreciated by the skilled artisan, the valve should be fabricated of material that is sufficiently flexible and resilient to enable to valve to return to a substantially closed, sealed position when the aerosol generator is disconnected. Thus, the valve at the aerosol inlet port enables a substantially constant pressure to be maintained within the ventilatory circuit even when the aerosol generator is not attached to the adaptor. Advantageously, the presence of the valve and resultant ability to maintain substantially constant positive pressure enables the adaptor to serve as a point of access, allowing safe application of catheters or surgical and diagnostic devices such as fiberoptic scopes to patients under ventilatory support, without interrupting such breathing support. The catheters may be cleaning catheters used to clean the upper or lower airways, nebulizing catheters to deliver aerosolized drugs as well as other substances or conduits to deliver liquid drugs as well as other substances to the airways. The adaptor can also include a removable cap to seal the aerosol inlet port when the port is not in use.

In certain embodiments, the adaptor can further include a one-way valve at the aerosol outlet port. The one-way valve can be fabricated of flexible, resilient material that may be the same or different from the material used to fabricate the valve at the aerosol inlet port. The one-way valve at the aerosol outlet port can be included to reduce or prevent any reverse aerosol flow that might occur during excessive expirations.

In certain embodiments, some of which are depicted in FIGS. 1A-4, the ventilation gas flow channel is adapted to form a chamber through which passes the aerosol flow channel. In such embodiments, the walls defining the aerosol flow channel extend beyond the ventilation gas flow channel as defined by the ventilation gas inlet and outlet ports. However, the length of the aerosol flow channel is also such that the aerosol outlet port is recessed from the patient interface port by a distance L2, so as to reduce the risk or incidence of expiratory resistance during controlled mechanical ventilation (CMV) or intermittent mechanical ventilation (IMV) and also sufficient to reduce or prevent the mixing of the ventilation flow with the flow of active agent. In certain embodiments, L2 is at least 2 mm. In certain embodiments designed for neonatal use, the aerosol outlet port is recessed from the patient interface port by at least about 8 millimeters (L2, FIG. 2B), with the chamber volume in the recess being at least about 1.4 milliliters. In certain embodiments designed for older infants, children or adults, the aerosol outlet port can be further recessed from the patient interface port, e.g., by at least about 9, 10, 11, 12, 13, 14, 15 or 16 millimeters, with concomitantly increased chamber volume in the recess, e.g., at least about 1.5, 1.6, 1.7, 1.8, 1.9, 2.0, 2.1, 2.2, 2.3, 2.4, 2.5, 2.6, 2.7, 2.8, 2.9 or 3.0 milliliters. In other embodiments, L2 is in a range of about 4 millimeters to about 8.5 millimeters.

The ventilatory circuit adaptor of the present invention can be made from any material suitable for the delivery of the substances described herein, e.g., polymers, metals, or composite materials. It is preferred that the materials are capable of being sterilized. The adaptors can be manufactured by methods known in the art, such as, for example, injection molding.

The ventilatory circuit adaptor of the present invention can be used in any ventilatory circuit to adapt it for use with an aerosol generator. The aerosol generator is introduced into the circuit via the adaptor. The aerosol generator may be directly or indirectly connected to the adaptor, e.g., via tubing, as would be understood by the skilled artisan. Any type of nebulizer or aerosol generator may be used. For instance, the aerosol generator can be an ultrasonic nebulizer or vibrating membrane nebulizer or vibrating screen nebulizer. Typically, jet nebulizers are not employed although the present methods can be adapted to all types of nebulizers or atomizers. In one embodiment, the aerosol generator is an Aeroneb® Professional Nebulizer (Aerogen Inc., Mountain View, Calif., USA). In another embodiment, the aerosol generator is a capillary aerosol generator, an example of which is a soft-mist generator by Philip Morris USA, Inc. Richmond, Va. (see U.S. Pat. Nos. 5,743,251 and 7,040,314; T. T. Nguyen, K. A. Cox, M. Parker and S. Pham (2003) Generation and Characterization of Soft-Mist Aerosols from Aqueous Formulations Using the Capillary Aerosol Generator, J. Aerosol Med. 16:189).

In certain embodiments, the adaptor can be used with a conduit inserted into the aerosol inlet port, through the aerosol flow channel and out the patient interface directly into the patient's nose (e.g., via nasal prongs or nasal tube) or mouth (e.g., via endotracheal tube) such that an active agent is provided in a liquid form or an aerosol form via the conduit.

The ventilation circuit further comprises a patient interface, which is selected to accommodate the type of ventilatory support to be administered. Invasive applications such as controlled, assisted or intermittent mandatory ventilation will utilize an endotracheal or tracheostomy tube as the patient interface. Non-invasive applications such as CPAP or BI-PAP may utilize nasal prongs or nasopharyngeal tubes, or a mask that covers the nose or both the nose and mouth, as the patient interface. In certain embodiments, the patient interface is connected directly to the adaptor. In other embodiments, a length of tubing may be introduced between the adaptor and the patient interface.

Thus, in practice, the system of the invention is utilized by establishing the patient on respiratory ventilation utilizing a circuit that includes the adaptor, introducing one or more active agents into the aerosol generator attached to the adaptor, and delivering to the patient through the adaptor a flow of the aerosolized active agent. The actual dosage of active agents will of course vary according to factors such as the extent of exposure and particular status of the subject (e.g., the subject's age, size, fitness, extent of symptoms, susceptibility factors, and the like). By “effective dose” herein is meant a dose that produces effects for which it is administered. The exact dose will be ascertainable by one skilled in the art using known techniques. In one exemplary embodiment, the effective dose of pulmonary surfactant for delivery to a patient by the present methods will be from about 2 mg/kg surfactant total phospholipid (TPL) to about 175 mg/kg surfactant TPL. The length of treatment time will also be ascertainable by one skilled in the art and will depend on dose administered and delivery rate of the active agent. For example, in embodiments wherein the delivery rate of aerosol to a patient is about 0.6 mg/min, greater than 100 mg of aerosol can be delivered in less than a 3 hour time frame. It will be understood by the skilled practitioner that a lower delivery rate will correspond to longer administration times and a higher delivery rate will correspond to shorter times. Similarly, a change in dose will affect treatment time.

Another aspect of the invention is an improvement in a method of delivery of an aerosolized active agent with concomitant positive pressure ventilation to a patient, wherein the improvement comprises diverting a portion of pressurized ventilation gas directed to the patient and combining it with a concentrated aerosolized active agent in a chamber and using the portion of the pressurized ventilation gas as a carrier (sheath) gas for delivery of the aerosolized active agent to the patient, thereby creating an auxiliary circuit for a carrier gas and aerosol delivery to a patient. It should be understood that the auxiliary circuit described in detail below can be used with any device or adaptor which enables delivery of a combination of a ventilation air and aerosol flows to a patient.

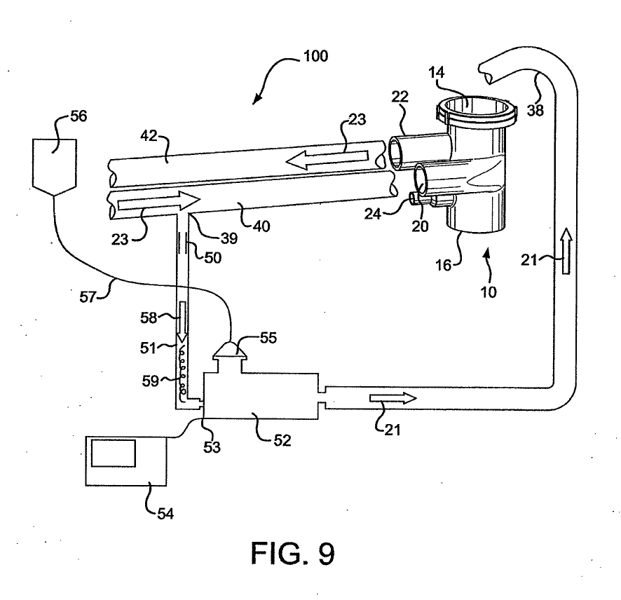

In yet another embodiment, the adaptor of the invention can be used in a novel aerosol delivery system. The combination of the adapter and the ventilation circuit described above creates a Proximal Aerosol Delivery System (PADS) 100 as exemplified in FIGS. 9-11. In the PADS, an auxiliary circuit is created for diverting a portion of the inspiratory ventilation flow to the aerosol entrainment chamber (AEC) to be used as a carrier or sheath gas for delivery of aerosolized active agent to the regulator. Advantageously, the AEC collects a concentrated aerosolized active agent which is then diluted with the sheath gas to the desired concentration. Thus, the sheath gas plays a dual role as a transporter and a diluent of the aerosolized active agent.

PADS 100 comprises an inspiratory arm 40 equipped with a T-connector 39. The T-connector 39 allows directing a predetermined portion of the flow from the ventilation circuit to the sheath gas tube 51. The amount of the ventilation air diverted to the sheath gas tube 51 is selected based on patient's PIF (2-5 L/min for newborns, 6-20 L/min for pediatric population and 20-30 L/min for adults). The sheath gas tube 51 has a flow restrictor 50. The sheath gas tube 51 with the flow restrictor 50 assures delivery of appropriate air flow to an aerosol entrainment chamber (AEC) 52. The sheath gas flow is equal to or higher than the patient's PIF and is regulated by a flow restrictor. The sheath gas flow is preferably within the range of 2-5 L/min for neonatal population and respectively higher for pediatric (e.g., 6-20 L/min) and adult populations (e.g., 20-60 L/min). In another variant, a built-in air flow regulator can be used in place of a flow restrictor for adjusting the sheath gas flow. In such case, the built-in air flow regulator is located in the AEC.

The sheath gas tube 51 can be connected to the inspiratory arm 40 of the ventilation circuit before or after a heater/humidifier (not shown). The placement of the sheath gas tube connector depends on the type of aerosol delivered to the patient. If the aerosol generated by the nebulizer is relatively dry and there is a risk for particles growth in the humidified environment, the sheath gas tube connector will be placed before the heater/humidifier. If the aerosol generated by the nebulizer is relatively wet and there is not a risk for additional particles growth in the humidified environment, the sheath gas connector can be placed after the heater/humidifier.

The inspiratory arm 40 is adapted to deliver the balance of the ventilation flow 23 to the adaptor 10 via the inspiratory flow port 20 as described above.

PADS 100 also comprises an expiratory arm 42 equipped with an exhalation filter (not shown). The exhalation filter has satisfactory capacity in order to prevent aerosol from reaching a PEEP valve and/or ambient air in the ‘bubble CPAP’ circuit set-up. The expiratory arm 42 is connected with the adaptor 10 via the expiratory flow port 22 and is adapted to remove ventilation air flow 23 from the adaptor 10.

The adaptor 10 (or 110) is connected to the inspiratory arm 40, and the expiratory arm 42 via inspiratory flow port 20 and expiratory flow port 22 respectively. The adaptor assures appropriate separation of ventilator flows directing undiluted aerosol towards patient.

The purpose of the AEC 52 is to provide maximal aerosol entrainment and high aerosol concentration to the adaptor 10. The AEC 52 may have a built-in flow regulator for sheath gas flow adjustment.

An aerosol generator 55 is located proximate to or connected with the AEC 52. It should be understood that any type of aerosol generator including, for example, mesh vibrating, jet or capillary aerosol generators, can be used in this invention.

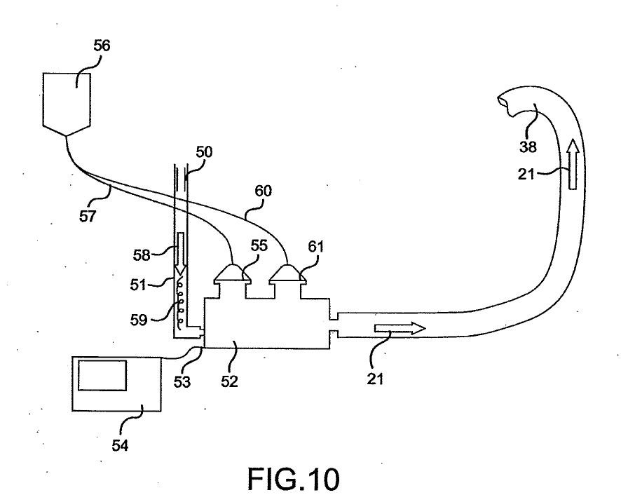

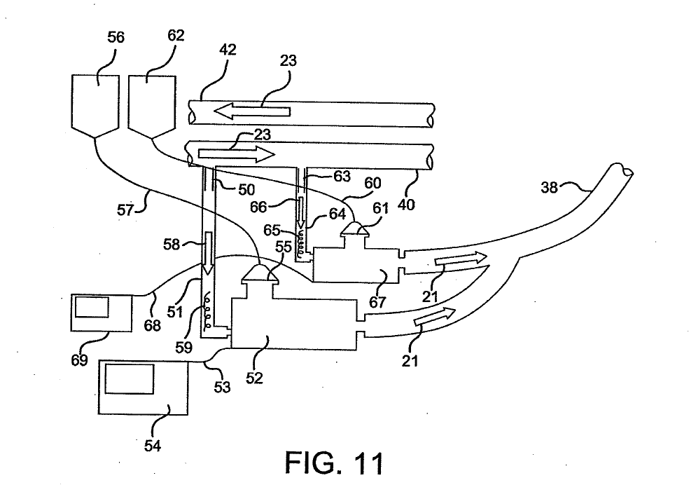

A drug reservoir 56 is connected with the aerosol generator 55 by means of a drug feeding line 57. The drug reservoir 56 and the feeding line assure drug supply to the aerosol generator, whenever nebulization is required including continuous supply. It should be understood that multiple drug reservoirs containing different drugs or reservoirs containing auxiliary substances other than drugs, e.g., pharmaceutically acceptable carriers together with multiple feeding lines, can be provided as needed (see, for example FIG. 11). Also, multiple aerosol generators can be used. An exemplary embodiment of such multiple aerosol generators is shown in FIG. 10, wherein a first aerosol generator 55 and a second aerosol generator 61 are connected to a drug reservoir 56 via first drug feeding line 57 and a second drug feeding line 60 respectively. In certain embodiments, the feeding line is eliminated and the drug reservoir is connected directly with the aerosol generator.

A heating device 59 as shown in FIGS. 9 and 10 is located within the sheath gas tube 51 and is used to heat the sheath gas 58 flowing though the sheath gas tube 51 before the entrance to the AEC 52. The heating device is optional. It can be used for delivery of a heated air/aerosol mixture to a patient. Heating of the sheath gas can also decrease potential particle growth as the sheath gas is not humidified.

As shown in FIG. 11, two drug reservoirs 56 and 62 are connected via drug feeding lines 57 and 60 to respective Aerosol Entrainment Chambers 52 and 67. The auxiliary circuits are formed via two T-connectors and flow restrictors 50 and 63 allowing diverting a portion of the inspiratory ventilation gas into sheath gas tubes 51 and 64 to a respective AEC 52 and 67 for contacting with the aerosolized drug. Connecting conduits 53 and 68 are connecting each AEC with a corresponding control unit 54 and 69, wherein each control unit can have a free standing or a built-in patient interface. Heating devices 59 and 65 are located within the sheath gas tube 51 and 64 respectively. The aerosol flow 21 is combined at a junction located in the aerosol tube 38.

AECs and drug reservoirs can be made of polycarbonate or materials known in the art suitable for operating at temperatures and pressures in the range of 18-40° C. and 5-60 cmH₂O.

An aerosol tube 38 is adopted to carry an entrained aerosol 21 from the AEC 52 to the aerosol inlet port 14. The length of the aerosol tube 38 can be selected to achieve optimal delivery based on the type of aerosol and characteristics of aerosol generators as known in the art. In certain embodiments, the AEC 52 is connected directly with the port 14 without the aerosol tube 38. Any known connector proving an appropriate seal can be used for this purpose In certain embodiments, the length of aerosol tube 38 does not exceed 20 cm. Preferably, the aerosol tube 38 is expandable to secure the optimized placement of the nebulizer, for example, as close to the patient as possible but in comfortable location to avoid restriction of any nursing procedures and allow patient for some head motion. Expandable tubes will help avoid sharp angle creation and thus avoid potential aerosol deposition within the delivery system.

The aerosol tube can be equipped with an optional expandable aerosol reservoir (not shown). This reservoir is a balloon with a volume equal to or as close as possible to a patient's tidal volume and with compliance equalizing PIF. During inspiration, the patient will be breathing in aerosol without diluting it as described above, whereas during exhalation the balloon will refill with aerosol up to the volume of tidal volume or similar and thus limit the aerosol losses to the expiratory arm of the circuit. The resistance of the balloon will maintain desired pressure within the ventilator system. During the phase following inspiration, the patient will inhale optimized highly concentrated aerosol from the balloon as it will be pushed away by elastic forces. This system will limit losses of the drug during exhalation. The size of the balloon depends on the patient's tidal volume and can differ for particular age groups.

A control unit 54 is located outside a patient bed (not shown). The control unit 54 has a user interface allowing for input/output of relevant information, e.g., patient weight. Any suitable control unit can be used in this invention. A patient's weight determines PIF which is matched with sheath gas flow. The control unit 54 is in communication with the aerosol generator 55 and the AEC 52 through a wire 53 or wirelessly (e.g., bluetooth technology).

Advantages of PADS as compared to the existing aerosol delivery models include (a) eliminates aerosol dilution by high ventilator gas flows within ventilator circuits, (b) eliminates additional sources for sheath gas flow or aerosol flow, and (c) proximal placement to a patient interface and thus reduction of potential drug losses within the PADS. Moreover, none of the PADS components increase dead space. Distant location of the control unit makes device operations much easier.

PADS can be used with different modes of ventilation including but not limiting to CPAP, IMV, and synchronized intermittent mechanical ventilation (SIMV). A simple version of PADS without a built-in flow regulator can operate on IMV/SIMV mode based on this same relative increase of the sheath gas flow through AEC driven by the increased flow or pressure within the ventilation circuit. Thus, the increased sheath gas flow will deliver more aerosol through the adaptor towards the patient during inhalation. A more complex version of PADS with a built-in flow generator will increase the flow of sheath gas based on a mechanism triggered by a patient. Such triggering mechanism can be based, for example, on Grasbay capsule sensing diaphragm motion or Electric Activity of the Diaphragm (EAdi) [12] which is clinically known as Neuronal Adjusted Ventilation (NAVA) sensing the phrenic and diaphragm nerve impulses. In such case the signals can be analyzed in a microprocessor controlling the flow meter within the AEC and sheath gas flow can be adjusted accordingly. In both scenarios described above, the nebulizer is operating continuously generating aerosol all the time. The aerosol generator can also be controlled based on the patient triggering mechanism. Again, the impulses based on NAVA technology could activate generation of aerosol before a patient is starting inspiration due to signal analysis by the microprocessor built in within AEC. The aerosol generator activation can be supported with the increased sheath gas flow as described above. The end of inspiration as well as aerosol generation can be determined based on the strength of the neuronal signal as described by NAVA.

The invention will be illustrated in more detail with reference to the following Examples, but it should be understood that the present invention is not deemed to be limited thereto.

### EXAMPLES

### Example 1

### Oxygen Dilution by Different Adaptor Designs

This protocol was designed to characterize the aerosol dilution effect of three different ventilation circuit adaptor adaptors for use with CPAP: a) the adaptor as described by U.S. Pat. No. publication 2006/0120968 to Niven et al. (adaptor 1); b) a ‘high resistant adaptor’ (adaptor 2 as shown in FIGS. 1A, 2A-4, 10 mm aerosol flow tube (L1 in FIG. 2B)); and c) a ‘low resistant adaptor’ (adaptor 3 as shown in FIGS. 1A, 2A-4, 5-6 mm aerosol flow tube (L1 in FIG. 2B)). In order to measure the dilution of aerosol, gases with two different concentrations of oxygen were used: 100% oxygen gas for aerosol flow and 21% oxygen gas for CPAP flow. The adaptors were tested under different CPAP flow conditions (6, 8, 10 and 12 L/min), and different steady state, potential inspiratory flows (0.3, 1.04, 3.22 and 5.18 L/min). The aerosol flow was constant at 3 L/min, the CPAP pressure maintained at 5 cm H₂O for all tested conditions.

The CPAP ventilation circuit was based on the Infant Star additional blended gas source with a flow meter. One end of the inspiratory limb of the circuit was connected to the blended gas flow meter and the other end to the inspiratory port of the tested ventilation circuit adaptor. The expiratory limb of the circuit was connected to the expiratory port of adaptor and the other end to a 5 cm H₂O PEEP valve. The ET tube port of the tested adaptor was connected to a rotameter through a ‘T’ connector. The oxymeter was connected to the circuit via this ‘T’ connector. A pressure manometer was connected to the adaptor via the pressure monitoring port. The oxymeter and pressure manometer were calibrated prior the initiation of the experiment. The oxygen tube was connected to the flow meter of the oxygen source and the other end to the aerosol port of the adaptor mimicking the aerosol flow. There were 5 recordings of every measurement done, 10 seconds apart. Collected data represent the oxygen concentration, and are presented as dilution factor value calculated using the equation:

Y=x−21%/79%

The results are presented as dilution factor values in Table 1. Both the adaptor 1 and the adaptor 2 (high resistance adaptor) showed no relationship between the different CPAP flows and the different inspiratory flows, i.e., no dilution was observed at any tested combination. Whenever inspiratory flow exceeded aerosol flow (i.e., was larger than approximately 3 L/min), a dilution effect was observed, as was expected. The adaptor 2 demonstrated somewhat better results for the condition when inspiratory flow was equal to aerosol flow. The adaptor 3 (low resistant CPAP adaptor) did not perform as well as the other two adaptors. A significant dilution effect was observed with CPAP flows higher than 4 L/min in the adaptor 3. The greatest dilution effect was noted for a CPAP flow of 12 L/min with a 0.8 dilution effect, compared to almost no dilution with the other two adaptors.

Overall, the design of the adaptors 2 and 3 is much different than the design of the adaptor 1. The inner volumes of both adaptors 2 and 3 are similar to the inner volume of the standard ‘Y’ connector, which allows for much safer use in combination with any type of breathing support. These adaptors can be used interchangeably for aerosol delivery under different ventilatory support conditions or just for ventilation during interim periods in aerosol therapy.

In summary, in this study, the adaptor 2 was superior in comparison to other two tested adaptors in introducing and directing undiluted oxygen towards the patient's interface due to the selection of L1.

TABLE 1

| Prior Art Adaptor - Adaptor1 | | | | | High Res. Adaptor - Adapto2 | | |
|---|---|---|---|---|---|---|---|
| CPAP Flow L/min | | | | | CPAP Flow L/min | | |
| 4 | 6 | 8 | 10 | 12 | 4 | 6 | 8 |

Insp Flow 0.3 L/min

| | | | | | | | | |
|---|---|---|---|---|---|---|---|---|
| #1 | 1 | 0.98734 | 0.98734 | 0.9747 | 0.98734 | 0.9873 | 1 | 1 |
| #2 | 1 | 1 | 0.97468 | 0.9873 | 0.98734 | 1 | 1.01266 | 1 |
| #3 | 1 | 0.98734 | 0.98734 | 0.9873 | 0.98734 | 0.9873 | 1 | 1 |
| #4 | 1 | 1 | 0.97468 | 0.9873 | 0.98734 | 0.9873 | 1 | 0.98734 |
| #5 | 0.987342 | 1 | 0.98734 | 0.9873 | 0.98734 | 0.9873 | 1 | 1 |
| mean | 0.997468 | 0.99494 | 0.98228 | 0.9848 | 0.98734 | 0.9899 | 1.00253 | 0.99747 |
| SD | 0.005661 | 0.00693 | 0.00693 | 0.0057 | 1.2E−16 | 0.0057 | 0.00566 | 0.00566 |

Insp Flow 1.04 L/min

| | | | | | | | | |
|---|---|---|---|---|---|---|---|---|
| #1 | 0.987342 | 0.97468 | 0.97468 | 0.9873 | 0.98734 | 0.9873 | 0.98734 | 0.97468 |
| #2 | 0.987342 | 0.97468 | 0.98734 | 0.9873 | 0.98734 | 0.9747 | 0.97468 | 0.98734 |
| #3 | 0.974684 | 0.96203 | 0.97468 | 0.9873 | 0.98734 | 0.9873 | 0.98734 | 0.98734 |
| #4 | 0.974684 | 0.97468 | 0.97468 | 0.9873 | 0.98734 | 0.9747 | 0.98734 | 0.98734 |
| #5 | 0.974684 | 0.97468 | 0.97468 | 0.9873 | 0.98734 | 0.9747 | 0.97468 | 0.98734 |
| mean | 0.979747 | 0.97215 | 0.97722 | 0.9873 | 0.98734 | 0.9797 | 0.98228 | 0.98481 |
| SD | 0.006933 | 0.00566 | 0.00566 | 1E−16 | 1.2E−16 | 0.0069 | 0.00693 | 0.00566 |

Insp Flow 3.22 L/min

| | | | | | | | | |
|---|---|---|---|---|---|---|---|---|
| #1 | 0.936709 | 0.93671 | 0.93671 | 0.9367 | 0.92405 | 0.9873 | 0.98734 | 0.98734 |
| #2 | 0.924051 | 0.94937 | 0.93671 | 0.9241 | 0.91139 | 0.9873 | 0.98734 | 0.98734 |
| #3 | 0.936709 | 0.94937 | 0.93671 | 0.9367 | 0.91139 | 1 | 0.98734 | 0.98734 |
| #4 | 0.924051 | 0.94937 | 0.92405 | 0.9367 | 0.92405 | 1 | 0.98734 | 0.98734 |
| #5 | 0.936709 | 0.93671 | 0.93671 | 0.9241 | 0.92405 | 1 | 0.98734 | 1 |
| mean | 0.931646 | 0.9443 | 0.93418 | 0.9316 | 0.91899 | 0.9949 | 0.98734 | 0.98987 |
| SD | 0.006933 | 0.00693 | 0.00566 | 0.0069 | 0.00693 | 0.0069 | 1.2E−16 | 0.00566 |

Insp Flow 5.18 L/min

| | | | | | | | | |
|---|---|---|---|---|---|---|---|---|
| #1 | 0.696203 | 0.67089 | 0.6962 | 0.6962 | 0.68354 | 0.5949 | 0.72152 | 0.78481 |
| #2 | 0.696203 | 0.67089 | 0.6962 | 0.6835 | 0.68354 | 0.5949 | 0.72152 | 0.78481 |
| #3 | 0.683544 | 0.6962 | 0.6962 | 0.6835 | 0.68354 | 0.6203 | 0.73418 | 0.77215 |
| #4 | 0.696203 | 0.68354 | 0.68354 | 0.6835 | 0.6962 | 0.5949 | 0.73418 | 0.77215 |
| #5 | 0.683544 | 0.6962 | 0.68354 | 0.6835 | 0.68354 | 0.5823 | 0.73418 | 0.77215 |
| mean | 0.691139 | 0.68354 | 0.69114 | 0.6861 | 0.68608 | 0.5975 | 0.72911 | 0.77722 |
| SD | 0.006933 | 0.01266 | 0.00693 | 0.0057 | 0.00566 | 0.0139 | 0.00693 | 0.00693 |

| High Res. Adaptor - Adapto2 | | Low Res. Adaptor - Adaptor 3 | | | | |
|---|---|---|---|---|---|---|
| CPAP Flow L/min | | CPAP Flow L/min | | | | |
| 10 | 12 | 4 | 6 | 8 | 10 | 12 |

Insp Flow 0.3 L/min

| | | | | | | | |
|---|---|---|---|---|---|---|---|
| #1 | 0.98734 | 0.98734 | 0.98734 | 0.94937 | 0.92405 | 0.86076 | 0.79747 |
| #2 | 1 | 0.98734 | 0.98734 | 0.94937 | 0.89873 | 0.86076 | 0.79747 |
| #3 | 0.98734 | 0.98734 | 0.98734 | 0.94937 | 0.89873 | 0.86076 | 0.79747 |
| #4 | 0.98734 | 0.98734 | 0.98734 | 0.93671 | 0.91139 | 0.86076 | 0.79747 |
| #5 | 1 | 0.98734 | 0.98734 | 0.94937 | 0.88608 | 0.8481 | 0.78481 |
| mean | 0.99241 | 0.98734 | 0.98734 | 0.94684 | 0.9038 | 0.85823 | 0.79494 |
| SD | 0.00693 | 1.2E−16 | 1.2E−16 | 0.00566 | 0.01443 | 0.00566 | 0.00566 |

Insp Flow 1.04 L/min

| | | | | | | | |
|---|---|---|---|---|---|---|---|
| #1 | 0.98734 | 0.97468 | 0.98734 | 0.96203 | 0.91139 | 0.8481 | 0.79747 |
| #2 | 0.98734 | 0.98734 | 0.97468 | 0.94937 | 0.89873 | 0.86076 | 0.79747 |
| #3 | 0.98734 | 0.98734 | 0.98734 | 0.96203 | 0.89873 | 0.86076 | 0.77215 |
| #4 | 0.98734 | 0.98734 | 0.97468 | 0.96203 | 0.91139 | 0.8481 | 0.79747 |
| #5 | 0.97468 | 0.98734 | 0.98734 | 0.96203 | 0.91139 | 0.8481 | 0.79747 |
| mean | 0.98481 | 0.98481 | 0.98228 | 0.95949 | 0.90633 | 0.85316 | 0.79241 |
| SD | 0.00566 | 0.00566 | 0.00693 | 0.00566 | 0.00693 | 0.00693 | 0.01132 |

Insp Flow 3.22 L/min

| | | | | | | | |
|---|---|---|---|---|---|---|---|
| #1 | 0.98734 | 0.98734 | 0.94937 | 0.89873 | 0.83544 | 0.77215 | 0.68354 |
| #2 | 0.98734 | 0.97468 | 0.93671 | 0.88608 | 0.8481 | 0.78481 | 0.68354 |
| #3 | 0.98734 | 0.97468 | 0.94937 | 0.88608 | 0.8481 | 0.77215 | 0.68354 |
| #4 | 0.98734 | 0.97468 | 0.94937 | 0.88608 | 0.8481 | 0.77215 | 0.68354 |
| #5 | 0.97468 | 0.98734 | 0.94937 | 0.88608 | 0.8481 | 0.77215 | 0.6962 |
| mean | 0.98481 | 0.97975 | 0.94684 | 0.88861 | 0.84557 | 0.77468 | 0.68608 |
| SD | 0.00566 | 0.00693 | 0.00566 | 0.00566 | 0.00566 | 0.00566 | 0.00566 |

Insp Flow 5.18 L/min

| | | | | | | | |
|---|---|---|---|---|---|---|---|
| #1 | 0.79747 | 0.78481 | 0.75949 | 0.70886 | 0.67089 | 0.62025 | 0.59494 |
| #2 | 0.81013 | 0.78481 | 0.75949 | 0.70886 | 0.67089 | 0.63291 | 0.58228 |
| #3 | 0.79747 | 0.78481 | 0.75949 | 0.6962 | 0.65823 | 0.62025 | 0.58228 |
| #4 | 0.81013 | 0.79747 | 0.74684 | 0.6962 | 0.65823 | 0.62025 | 0.58228 |
| #5 | 0.81013 | 0.79747 | 0.74684 | 0.70886 | 0.65823 | 0.62025 | 0.58228 |
| mean | 0.80506 | 0.78987 | 0.75443 | 0.7038 | 0.66329 | 0.62278 | 0.58481 |
| SD | 0.00693 | 0.00693 | 0.00693 | 0.00693 | 0.00693 | 0.00566 | 0.00566 |

### Example 2

### Resistance Measurements of Different Adaptor Designs

The purpose of this study was to evaluate the operational characteristics of different ventilation circuit adaptors used for aerosol introduction into the CPAP ventilation circuit at the level of a ‘Y’ connector. Operational characteristics were assessed based on the resistance values of different adaptors tested under typical ventilation conditions for the potential targeted neonatal population.

The protocol was designed to characterize the operational characteristics of three different ventilation circuit adaptors and a standard ‘Y’ connector under dynamic flow conditions as intermittent mechanical ventilation (IMV): a) the adaptor as described by US patent publication 2006/0120968 to Niven et al. (the adaptor 1); b) a ‘high resistant CPAP adaptor’ (the adaptor 2 as shown in FIGS. 1A, 2A-4, 10 mm aerosol flow tube); c) a ‘low resistant adaptor’ (the adaptor 3 as shown in FIGS. 1A, 2A-4, 5-6 mm aerosol flow tube); and d) a ‘standard Y connector’ (the adaptor 4). These CPAP adaptors were tested under two different inspiratory flow conditions (approximately 1 and 3 L/min respectively). The operational characteristics of different adaptors were based on resistance measurements performed by airway manometry and pneumotachography.

The ventilator circuit was based on the Harvard small animal ventilator. One end of the inspiratory limb of the circuit was connected to the inspiratory port of the ventilator and the other end to the inspiratory port of the tested ventilation circuit adaptor. The expiratory limb of the circuit was connected to the expiratory port of the adaptor and the other end to the expiratory port of the Harvard ventilator. A pressure manometer was connected to the adaptor via the pressure monitoring port. The pressure manometer was calibrated prior the initiation of the experiment. The aerosol port of the adaptor was securely closed. There was 1 recording for every measurement done based on the PEDS calculations from at least 10 breathing cycles. Data represent the mean and standard error of the mean (SEM) values of inspiratory, expiratory, and total resistance.

The results are presented as mean and SEM values for total, inspiratory and expiratory resistance in Table 2. None of the tested adaptors showed higher resistance values (within 10%) compared to the ‘standard Y connector’ (the adaptor 4), which served as a reference for this test. In fact, the ‘high resistant adaptor’ (the adaptor 2) had lower resistance values measured under two different inspiratory flow conditions than the ‘standard Y connector’.

TABLE 2

| | PIF = 1.3-1.4 mL/min | | | | | | PIF = 2.9-3.2 mL/min | | | | | |
|---|---|---|---|---|---|---|---|---|---|---|---|---|
| | Resistance mL/cmH₂0 | | | | | | Resistance mL/cmH₂0 | | | | | |
| | Inspiratory | | Expiratory | | Total | | Inspiratory | | Expiratory | | Total | |
| Adaptor | mean | SEM | mean | SEM | mean | SEM | mean | SEM | mean | SEM | Mean | SEM |
| #1 | 28.02 | 0.68 | 35.56 | 0.12 | 24.62 | 0.06 | 33.58 | 0.23 | 57 | 0.7 | 39.98 | 1.46 |
| #2 | 27.9 | 0.44 | 32.08 | 0.04 | 25.34 | 0.07 | 26 | 0.22 | 49.78 | 0.28 | 30.43 | 0.19 |
| #3 | 33.63 | 0.28 | 35.55 | 0.13 | 27.11 | 0.18 | 31.57 | 0.18 | 55.17 | 0.57 | 38.74 | 0.21 |
| #4 | 32.04 | 0.28 | 30.26 | 5.5 | 26.61 | 0.7 | 29.98 | 0.4 | 55.39 | 0.33 | 36.46 | 0.27 |

### Example 3

### Preclinical Study

A preclinical study on preterm lamb has been aimed on proving the efficacy of aerosolized lucinactant for inhalation for prevention of RDS, and has utilized an embodiment of the ventilatory circuit adaptor of the invention as shown in FIGS. 1A, 2A. Four preterm lambs with gestation age of 126-128 days were treated with CPAP after preterm delivery. Within 30 minutes after birth the aerosolized surfactant treatment was initiated. The adaptor has efficiently delivered aerosol to the animals without any noted adverse events.

While the invention has been described in detail and with reference to specific examples thereof, it will be apparent to one skilled in the art that various changes and modifications can be made therein without departing from the spirit and scope thereof.

What is claimed is:

1. A ventilation circuit adaptor, comprising:
an aerosol flow chamber having a first end, a second end opposite the first end, a first longitudinal axis, an inner wall spaced apart from and surrounding the first longitudinal axis, an aerosol chamber inlet port located at the first end and having a first chamber cross-sectional area, and a patient interface port located at the second end and having a second chamber cross-sectional area;
a ventilation gas flow chamber in fluid communication with the aerosol flow chamber and having a primary end, an other end spaced apart from the primary end, a second longitudinal axis, a ventilation gas inlet port located at the primary end, and a ventilation gas outlet port located at the other end,
wherein the second longitudinal axis is at least partially offset from the first longitudinal axis and at least partially encircles the first longitudinal axis, and
a funnel-shaped aerosol flow channel adapted to be inserted into and fixedly positioned in the aerosol flow chamber, the funnel-shaped aerosol flow channel having a first channel end, an other channel end opposite the first channel end, a channel longitudinal axis, an outer wall spaced apart from and surrounding the channel longitudinal axis, an aerosol channel inlet port located at the first channel end and having a first channel cross-sectional area, and a channel outlet port located at the second channel end and having a second channel cross-sectional area smaller than the first channel cross-sectional area,
wherein the channel longitudinal axis of the funnel-shaped aerosol flow channel is coaxial with the first longitudinal axis of the aerosol flow chamber when the funnel-shaped aerosol flow channel is fixedly positioned in the aerosol flow chamber.

2. A ventilation circuit adaptor as in claim 1, further comprising:
a positive interference seal on a portion of the outer wall of the funnel-shaped aerosol flow channel, the positive interference seal adapted to form a positive interference fit with a portion of the inner wall of the aerosol flow chamber.

3. A ventilation circuit adaptor as in claim 2, wherein the positive interference seal includes a ridge protruding from the portion of the outer wall of the funnel-shaped aerosol flow channel.

4. A ventilation circuit adaptor as in claim 1, further comprising:
at least one assembly alignment fixture adapted to prevent rotational movement of the funnel-shaped aerosol flow channel when it is fixedly positioned in the aerosol flow chamber.

5. A ventilation circuit adaptor as in claim 4, wherein the at least one assembly alignment fixture comprises:
at least one aperture or recess in the inner wall of the aerosol flow chamber, and
at least one snap-in catch on the outer wall of the funnel-shaped aerosol flow channel adapted to lock with the at least one aperture or recess.

6. A ventilation circuit adaptor as in claim 1, wherein the first chamber cross-sectional area is greater than the second chamber cross-sectional area.

7. A ventilation circuit adaptor as in claim 1, wherein the channel outlet port extends beyond the gas inlet port and the gas outlet port, and the second channel end is recessed from the patient interface port.

8. A ventilation circuit adaptor as in claim 7, wherein the second channel end is recessed from the patient interface port by a distance in a range of about 4 millimeters to about 8.5 millimeters.

9. A ventilation circuit adaptor as in claim 1, further comprising:
a pressure sensor port in fluid communication with the aerosol flow chamber below the gas inlet port and the gas outlet port.

10. A ventilation circuit adaptor as in claim 1, further comprising:
a removable stopper or cap tethered to an outer surface of the ventilation circuit adaptor and adapted to close the aerosol chamber inlet port.

11. A ventilation circuit adaptor as in claim 4, wherein the at least one assembly alignment fixture includes two or more assembly alignment fixtures circumferentially spaced apart from each other by about 60° to about 180°.

12. A ventilation circuit adaptor as in claim 1, further comprising:
a reducer having a second inner diameter smaller than a first inner diameter of the patient interface port,
wherein the reducer is adjacent to and in fluid communication with the patient interface port and is adapted to receive an aerosol flow from the patient interface port.

13. A ventilation circuit adaptor as in claim 12, wherein a portion of the reducer is connected to the inner wall near the second end of the aerosol flow chamber by a connecting technique selected from a group consisting of ultrasonic welding, gluing, and laser welding.

14. A ventilation circuit adaptor, comprising:
an aerosol flow chamber having an aerosol inlet port and a patient interface port, and defining an aerosol flow path from the aerosol inlet port to and through the patient interface port having a first inner diameter;
a ventilation gas flow chamber in fluid communication with the aerosol flow chamber and having a gas inlet port and a gas outlet port, and defining a ventilation gas flow path from the gas inlet port to and through the gas outlet port,
wherein the ventilation gas flow path is at least partially offset from the aerosol flow path and at least partially encircles the aerosol flow path, and
wherein the ventilation gas flow chamber forms a chamber that includes the gas inlet port, the gas outlet port and the patient interface port, wherein an aerosol flow channel is contained within the chamber and extends from the aerosol inlet port at one end of the chamber through the chamber to an aerosol outlet port within the chamber and is recessed from the patient interface port at the opposite end of the chamber, wherein the aerosol flow channel has a substantially uniform cross-sectional area and is of a sufficient length to extend beyond the gas inlet and outlet ports; and
a reducer having a second inner diameter smaller than the first inner diameter of the patient interface port,
wherein the reducer is adjacent to and in fluid communication with the patient interface port and is adapted to receive an aerosol flow from the patient interface port.

15. A ventilation circuit adaptor as in claim 14, wherein a portion of the reducer is connected to an inner wall of the chamber near the patient interface port by a connecting technique selected from a group consisting of ultrasonic welding, gluing, and laser welding.

16. A method for assembling a ventilation circuit adaptor, comprising the steps of:
providing an aerosol flow chamber having a first end, a second end opposite the first end, a first longitudinal axis, an inner wall spaced apart from and surrounding the first longitudinal axis, an aerosol chamber inlet port located at the first end and having a first chamber cross-sectional area, and a patient interface port located at the second end and having a second chamber cross-sectional area;
providing a ventilation gas flow chamber in fluid communication with the aerosol flow chamber and having a primary end, an other end spaced part from the primary end, a second longitudinal axis, a ventilation gas inlet port located at the primary end, and a ventilation gas outlet port located at the other end,
wherein the second longitudinal axis is at least partially offset from the first longitudinal axis and at least partially encircles the first longitudinal axis;
providing a funnel-shaped aerosol flow channel adapted to be inserted into and fixedly positioned in the aerosol flow chamber, the funnel-shaped aerosol flow channel having a first channel end, an other channel end opposite the first channel end, a channel longitudinal axis, an outer wall spaced apart from and surrounding the channel longitudinal axis, an aerosol channel inlet port located at the first channel end and having a first channel cross-sectional area, and a channel outlet port located at the second channel end and having a second channel cross-sectional area smaller than the first channel cross-sectional area,
wherein the channel longitudinal axis of the funnel-shaped aerosol flow channel is coaxial with the first longitudinal axis of the aerosol flow chamber when the funnel-shaped aerosol flow channel is fixedly positioned in the aerosol flow chamber;
inserting the funnel-shaped aerosol flow chamber into the aerosol flow chamber; and
fixedly positioning the funnel-shaped aerosol flow channel in the aerosol flow chamber so that the channel longitudinal axis of the funnel-shaped aerosol flow channel is coaxial with the first longitudinal axis of the aerosol flow chamber.

17. A method for assembling a ventilation circuit adaptor as in claim 16, comprising the further steps of:
providing a positive interference seal on a portion of the outer wall of the funnel-shaped aerosol flow channel, the positive interference seal adapted to form a positive interference fit with a portion of the inner wall of the aerosol flow chamber; and
forming the positive interface fit by the interference seal with the portion of the inner wall of the aerosol flow chamber.

18. A method for assembling a ventilation circuit adaptor as in claim 17, wherein the positive interference seal includes a ridge protruding from the portion of the outer wall of the funnel-shaped aerosol flow channel.

19. A method for assembling a ventilation circuit adaptor as in claim 16, comprising the further step of:
providing at least one assembly alignment fixture adapted to prevent rotational movement of the funnel-shaped aerosol flow channel when it is fixedly positioned in the aerosol flow chamber.

20. A method for assembling a ventilation circuit adaptor as in claim 19, wherein the at least one assembly alignment fixture comprises:
at least one aperture or recess in the inner wall of the aerosol flow chamber, and
at least one snap-in catch on the outer wall of the funnel-shaped aerosol flow channel adapted to lock with the at least one aperture or recess.

21. A method for assembling a ventilation circuit adaptor as in claim 20, comprising the further step of:
locking the at least one snap-in catch with the at least one aperture or recess.

22. A method for assembling a ventilation circuit adaptor as in claim 16, comprising the further step of:
providing a pressure sensor port in fluid communication with the aerosol flow chamber below the gas inlet port and the gas outlet port.

23. A method for assembling a ventilation circuit adaptor as in claim 16, comprising the further step of:
providing a removable stopper or cap tethered to an outer surface of the ventilation circuit adaptor and adapted to close the aerosol chamber inlet port.

24. A method for assembling a ventilation circuit adaptor as in claim 19, wherein the at least one assembly alignment fixture includes two or more assembly alignment fixtures circumferentially spaced apart from each other by about 60° to about 180°.

25. A method for assembling a ventilation circuit adaptor as in claim 16, comprising the further step of:
providing a reducer having a second inner diameter smaller than a first inner diameter of the patient interface port,
wherein the reducer is adjacent to and in fluid communication with the patient interface port and is adapted to receive an aerosol flow from the patient interface port.

26. A method for assembling a ventilation circuit adaptor as in claim 25, wherein a portion of the reducer is connected to the inner wall near the second end of the aerosol flow chamber by a connecting technique selected from a group consisting of ultrasonic welding, gluing, and laser welding.

27. A method for assembling a ventilation circuit adaptor, comprising the steps of:
providing an aerosol flow chamber having an aerosol inlet port and a patient interface port, and defining an aerosol flow path from the aerosol inlet port to and through the patient interface port having a first inner diameter;
providing a ventilation gas flow chamber in fluid communication with the aerosol flow chamber and having a gas inlet port and a gas outlet port, and defining a ventilation gas flow path from the gas inlet port to and through the gas outlet port,
wherein the ventilation gas flow path is at least partially offset from the aerosol flow path and at least partially encircles the aerosol flow path, and
wherein the ventilation gas flow chamber forms a chamber that includes the gas inlet port, the gas outlet port and the patient interface port, wherein an aerosol flow channel is contained within the chamber and extends from the aerosol inlet port at one end of the chamber through the chamber to an aerosol outlet port within the chamber and is recessed from the patient interface port at the opposite end of the chamber, wherein the aerosol flow channel has a substantially uniform cross-sectional area and is of a sufficient length to extend beyond the gas inlet and outlet ports;
providing a reducer having a second inner diameter smaller than the first inner diameter of the patient interface port,
wherein the reducer is adjacent to and in fluid communication with the patient interface port and is adapted to receive an aerosol flow from the patient interface port; and
connecting the reducer to an inner wall of the chamber near the patient interface port.

28. A method for assembling a ventilation circuit adaptor as in claim 27, comprising the further step of:
providing a pressure sensor port in fluid communication with the aerosol flow chamber below the gas inlet port and the gas outlet port.

29. A method for assembling a ventilation circuit adaptor as in claim 27, comprising the further step of:
providing a removable stopper or cap tethered to an outer surface of the ventilation circuit adaptor and adapted to close the aerosol chamber inlet port.

30. A method for assembling a ventilation circuit adaptor as in claim 27, wherein the way of connecting the reducer to the inner wall of the chamber near the patient interface port is selected from a group consisting of ultrasonic welding, gluing, and laser welding.
